(19) United States
(12) Patent Application Publication
(10) Pub. No.: US 2016/0271023 A1
(43) Pub. Date: Sep. 22, 2016

(54) COSMETIC COMPOSITION COMPRISING BI-MODAL EMULSION

(71) Applicant: DOW CORNING CORPORATION, Midland, MI (US)

(72) Inventors: Thomas Daniel BEKEMEIER, Birch Run, MI (US); Bethany K. JOHNSON, Midland, MI (US); Donald Taylor LILES, Midland, MI (US); Heidi Marie VANDORT, Sanford, MI (US); Brett Lee ZIMMERMAN, Frankenmuth, MI (US)

(21) Appl. No.: 14/442,263

(22) PCT Filed: Nov. 21, 2013
(86) PCT No.: PCT/US2013/071252

§ 371 Date: May 12, 2015

Related U.S. Application Data
(60) Provisional application No. 61/729,024, filed on Nov. 21, 2012.

(51) Int. Cl. A61K 8/06; A61Q 19/10; A61Q 1/02; A61Q 5/12; A61Q 17/04; A61K 8/81; A61Q 5/02; A61K 8/891; A61K 8/892; A61K 8/86; A61K 8/895; A61K 8/92; A61K 8/04; A61Q 15/00

(52) U.S. Cl. CPC A61K 8/062; A61K 8/068; A61K 8/046; A61K 8/042; A61K 8/064; A61Q 19/10; A61Q 1/02; A61Q 5/12; A61Q 17/04; A61Q 15/00; A61Q 5/02; A61K 8/891; A61K 8/892; A61K 8/86; A61K 8/895; A61K 8/922; A61K 8/8111

(57) ABSTRACT

Cosmetic compositions comprising bi-modal water continuous emulsions are disclosed. In particular, the cosmetic compositions comprise a bi-modal water continuous emulsion (E) comprising at least 70 weight percent of: a first dispersed phase containing a hydrophobic oil, wherein the hydrophobic oil is provided as a non-emulsified hydrophobic oil, and a second dispersed phase containing a silicone, wherein the silicone is provided from a water continuous silicone emulsion containing at least one surfactant. The cosmetic compositions further comprise at least one cosmetic ingredient (C). Optionally, the cosmetic ingredient (C) is in a cosmetically acceptable medium. Preparation methods and uses of the cosmetic compositions are also disclosed.

### CROSS-REFERENCE TO RELATED APPLICATIONS

This application claims priority to and all the advantages of U.S. Provisional Patent Application No. 61/729,024, filed on Nov. 21, 2012, the content of which is incorporated herein by reference.

### TECHNICAL FIELD

This disclosure relates to cosmetic compositions comprising an emulsion (E), and at least one cosmetic ingredient (C), in a cosmetically acceptable medium.

### BACKGROUND

Emulsions are a useful means compatibilising initially incompatible materials. Simple emulsions are usually at least composed of an external, continuous phase and an internal, dispersed phase and at least one surface active agent used to compatibilise the distinct phases. Emulsions of various ingredients may be used in cosmetic applications, where they allow incorporation of incompatible materials in a larger matrix such as vitamins in a skin care cream, or conditioning polymers in a shampoo.

There is a continuous need to provide for emulsions which lower the environmental impact of transport, by reducing the amount of water contained in the emulsion. There is a continuous need to provide for emulsions which are not detrimental to the health of consumers by reducing the amount of surfactant contained in the emulsion or by reducing the amount of biocide.

There is a continuous need to simplify the formulator's work by providing for emulsions which are easy to use, pourable, while at the same time containing a limited amount of unnecessary ingredients which allow larger latitude for useful ingredients. That is, limiting the amount of water to allow incorporation of more active ingredients such as dyes, care agents and the like.

There is an ongoing need for emulsions of conditioning polymers which provide for care of keratinous substrates.

### BRIEF SUMMARY OF THE INVENTION

The present invention relates to cosmetic compositions comprising bi-modal water continuous emulsions. In particular, the present disclosure relates to cosmetic compositions comprising a bi-modal water continuous emulsion (E) comprising at least 70 weight percent of: a first dispersed phase containing a hydrophobic oil, wherein the hydrophobic oil is provided as a non-emulsified hydrophobic oil, a second dispersed phase containing a silicone, wherein the silicone is provided from a water continuous silicone emulsion containing at least one surfactant; and at least one cosmetic ingredient (C), in a cosmetically acceptable medium.

The invention also relates to a process to prepare the cosmetic compositions and uses of said cosmetic compositions.

### DETAILED DESCRIPTION OF THE INVENTION

The present invention relates to cosmetic compositions comprising emulsions. In particular, the present disclosure relates to cosmetic compositions comprising a bi-modal water continuous emulsion (E) comprising at least 70 weight percent of: a first dispersed phase containing a hydrophobic oil, wherein the hydrophobic oil is provided as a non-emulsified hydrophobic oil, a second dispersed phase containing a silicone, wherein the silicone is provided from a water continuous silicone emulsion containing at least one surfactant; and at least one cosmetic ingredient (C), in a cosmetically acceptable medium.

The present bi-modal emulsions (E) are water continuous emulsions having two distinct dispersed phases. As used herein, “dispersed phase” refers to the water insoluble particles suspended in the continuous aqueous phase of the emulsion. The first dispersed phase contains a hydrophobic oil, which may be either an organic oil or a silicone. The independent second dispersed phase contains a silicone that is provided from a previously formed water continuous emulsion. Each dispersed phase may be characterized by its own average particle size distribution in the emulsion, in other words, the average particle size of the two independent dispersed phases demonstrate a “bi-modal” distribution.

### The First Dispersed Phase of Bi-Modal Emulsion (E)

The bi-modal emulsions contain a first dispersed phase containing a hydrophobic oil (designated herein as component (A)). The hydrophobic oil (A) in the first dispersed phase of the present bi-modal emulsion has not been pre-emulsified. In other words, the hydrophobic oils in the first dispersed phase are derived from neat or non-emulsified hydrophobic oils. The hydrophobic oil (A) may be selected from a) an organic oil, b) a silicone, or combinations thereof.

When the hydrophobic oil phase is considered to be an organic oil phase, it means the majority of this dispersed phase comprises organic compounds or organic polymers. The organic oil may be selected from hydrocarbons, esters, oils derived from natural fats or oils, organic polymers, or mixtures thereof.

Examples of suitable organic oil components include natural oils such as coconut oil; hydrocarbons such as mineral oil, paraffins and hydrogenated polyisobutene; fatty alcohols such as octyldodecanol; esters such as C12-C15 alkyl benzoate; diesters such as propylene dipelarganate; and triesters, such as glyceryl trioctanoate.

Examples of esters as suitable organic oil may have the structure QCO-OQ′ wherein QCO represents the carboxylic acid radical and wherein OQ′ is an alcohol residue. Examples of these esters include isotridecyl isononanoate, PEG-4 diheptanoate, isostearyl neopentanoate, tridecyl neopentanoate, cetyl octanoate, cetyl palmitate, cetyl ricinoleate, cetyl stearate, cetyl myristate, coco-dicaprylate/caprate, decyl isostearate, isodecyl oleate, isodecyl neopentanoate, isohexyl neopentanoate, octyl palmitate, dioctyl malate, tridecyl octanoate, myristyl myristate, octododecanol, or mixtures of octyldodecanol, acetylated lanolin alcohol, cetyl acetate, isododecanol, polyglyceryl-3-diisostearate, or mixtures thereof.

Examples of natural oils include castor oil, lanolin and lanolin derivatives, triisocetyl citrate, sorbitan sesquioleate, C10-18 triglycerides, caprylic/capric/triglycerides, coconut oil, corn oil, cottonseed oil, glyceryl triacetyl hydroxystearate, glyceryl triacetyl ricinoleate, glyceryl trioctanoate, hydrogenated castor oil, linseed oil, mink oil, olive oil, palm oil, castor oil, illipe butter, rapeseed oil, soybean oil, sunflower seed oil, pine oil, tallow, tricaprin, trihydroxystearin, triisostearin, trilaurin, trilinolein, trimyristin, triolein, tripalmitin, tristearin, walnut oil, wheat germ oil, cholesterol, or mixtures thereof.

The organic oil may further contain an organic polymer such as polybutenes or polyisobutylenes, polyacrylates, polystyrenes, polybutadienes, polyamides, polyesters, polyacrylates, polyurethanes, polysulfones, polysulfides, epoxy functional polymers, as well as copolymers or terpolymers containing these organic polymers, and mixtures of any of these.

Further suitable organic oils may be solid at room temperature, such as organic butters and organic waxes.

Examples of butters include shea butter, mango butter.

Examples of organic waxes include those selected from synthetic and natural origin such as mineral waxes, animal waxes, vegetal waxes, hydrogenated oils, fatty esters and glycerides which are solid at 25° C.

Examples of organic waxes include esters derived from a monovalent saturated C16-C60 alkanol and a saturated C8-C36 monocarboxylic acid, glycerol triesters of saturated linear C18-C40 carboxylic acids, candelilla wax, carnauba wax, beeswax, saturated linear C16-C18, C20, and C22-C40 carboxylic acids, hardened castor oil, ozokerite, polyethylene wax, microcrystalline wax, ceresin, lanolin wax, rice bran wax, montan wax, orange wax, lemon wax and paraffin wax.

The hydrophobic oil (A) may be selected from various silicone polymers. In this embodiment, the hydrophobic oil phase is considered to be a silicone oil phase, which means the majority of this dispersed phase comprises silicone polymers. As used herein, “silicone composition” refers to a composition containing at least one organopolysiloxane.

Organopolysiloxanes are polymers containing siloxy units independently selected from (R₃SiO_{1/2}), (R₂SiO_{2/2}), (RSiO_{3/2}), or (SiO_{4/2}) siloxy units, where R may be any organic group, alternatively R is a hydrocarbon group containing 1 to 30 carbons, alternatively R is an alkyl group containing 1 to 12 carbon atoms, or alternatively R is methyl or phenyl. These siloxy units are commonly referred to as M, D, T, and Q units respectively. Their molecular structures are listed below:

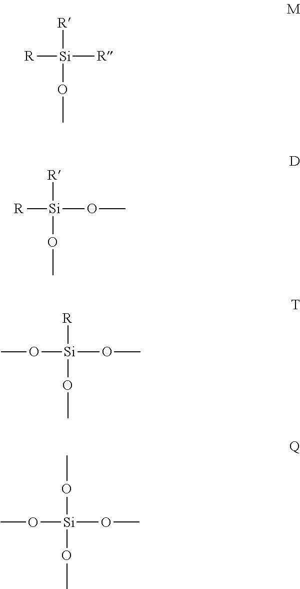

These siloxy units can be combined in various manners to form cyclic, linear, or branched structures. The chemical and physical properties of the resulting polymeric structures vary depending on the number and type of siloxy units in the organopolysiloxane.

The silicone composition may contain a single organopolysiloxane, or mixture of various organopolysiloxanes. In some instances, the mixture of organopolysiloxanes can react with each other to form higher molecular weight organopolysiloxanes. Such reactions are exemplified by condensation or hydrosilylation reactions.

The silicone composition may contain silicone fluids, silicone gums, silicone rubbers, silicone elastomers, silicone resins, silicone waxes, saccharide-siloxane polymer, vinyl polymer grafted with a carbosiloxane dendrimers or any combinations thereof.

The organopolysiloxane may be a trimethylsiloxy or hydroxy (SiOH) terminated polydimethylsiloxane. Trimethoxy end blocked polydimethysiloxanes have the formula Me₃SiO(Me₂SiO_{2/2})_{dp}Si Me₃ wherein the degree of polymerization (dp) is greater than 1, or alternatively the dp is sufficient to provide a kinematic viscosity that may range from 1 to 1,000,000 mm²/s (10⁻⁶ m²/s) at 25° C., or alternatively from 100 to 600,000 mm²/s (10⁻⁶ m²/s) at 25° C., or alternatively from 1000 to 600,000 mm²/s (10⁻⁶ m²/s) at 25° C.

When the silicone composition contains organopolysiloxanes components that can react via hydrosilylation, the silicone component contains:
b¹) an organopolysiloxane having at least two silicon-bonded alkenyl groups per molecule,
b²) an organohydrogensiloxane having at least two SiH groups per molecule, and
b³) a hydrosilylation catalyst.

The organopolysiloxane having at least two silicon-bonded alkenyl groups per molecule b¹) comprises at least two siloxy units represented by the formula

R²RₘSiO_{(4-1-m)/2}

wherein R is an hydrocarbon group containing 1 to 30 carbon atoms, R² is an alkenyl group containing 2 to 12 carbon atoms, and m is zero to 2. The R² alkenyl groups of Component b¹) are exemplified by vinyl, allyl, 3-butenyl, 4-pentenyl, 5-hexenyl, 6-heptenyl, 7-octenyl, 8-nonenyl, 9-decenyl, 10-undecenyl, 4,7-octadienyl, 5,8-nonadienyl, 5,9-decadienyl, 6, 11-dodecadienyl and 4,8-nonadienyl.

The R² alkenyl group may be present on any mono, di, or tri siloxy unit in the organopolysiloxane, for example: (R²R₂SiO_{1/2}), (R²RSiO_{2/2}), or (R²SiO_{3/2}); as well as in combination with other siloxy units not containing an R² substituent, such as (R₃SiO_{1/2}), (R₂SiO_{2/2}), (RSiO_{3/2}), or (SiO_{4/2}) siloxy units where R is a hydrocarbon containing 1 to 20 carbons, alternatively an alkyl group containing 1 to 12 carbons, alternatively an alkyl group containing 1 to 6 carbons or alternatively methyl; providing there are at least two R² substituents in the organopolysiloxane. The monovalent hydrocarbon group R having from 1 to 20 carbon atoms is exemplified by alkyl groups such as: methyl, ethyl, propyl, butyl, hexyl, octyl, and decyl; cycloaliphatic groups such as cyclohexyl; aryl groups such as phenyl, tolyl, and xylyl;, and aralkyl groups such as benzyl and phenylethyl.

Component b¹) may be selected from trimethylsiloxy-terminated polydimethylsiloxane-polymethylvinylsiloxane copolymers, vinyldimethylsiloxy-terminated polydimethylsiloxane-polymethylvinylsiloxane copolymers, trimethylsiloxy-terminated polydimethylsiloxane-polymethylhexenylsiloxane copolymers, hexenyldimethylsiloxy-terminated polydimethylsiloxane-polymethylhexenylsiloxane copolymers, trimethylsiloxy-terminated polymethylvinylsiloxane polymers, trimethylsiloxy-terminated polymethylhexenylsiloxane polymers, vinyldimethylsiloxy-terminated polydimethylsiloxane polymers, hexenyldimethylsiloxy-terminated polydimethylsiloxane polymers, or any combination thereof, each having a degree of polymerization of from 10 to 300, or alternatively having a viscosity at 25° C. of from 10 to 1000 mPa·s (10⁻³ Pa·s).

Component b²) is an organohydrogensiloxane having an average of greater than two silicon bonded hydrogen atoms per molecule. As used herein, an organohydrogensiloxane is any organopolysiloxane containing a silicon-bonded hydrogen atom (SiH).

Organohydrogensiloxanes are organopolysiloxanes having at least one SiH containing siloxy unit, that is at least one siloxy unit in the organopolysiloxane has the formula (R₂HSiO_{1/2}), (RHSiO_{2/2}), or (HSiO_{3/2}). Thus, the organohydrogensiloxanes useful in the present invention may comprise any number of (R₃SiO_{1/2}), (R₂SiO_{2/2}), (RSiO_{3/2}), (R₂HSiO_{1/2}), (RHSiO_{2/2}), (HSiO_{3/2}) or (SiO_{4/2}) siloxy units, providing there are on average at least two SiH siloxy units in the molecule. Component b²) can be a single linear or branched organohydrogensiloxane or a combination comprising two or more linear or branched organohydrogensiloxanes that differ in at least one of the following properties: structure, viscosity, average molecular weight, siloxane units, and sequence. There are no particular restrictions on the molecular weight of the organohydrogensiloxane, but typically the viscosity of the organohydrogensiloxane at 25° C. is from 3 to 10,000 mPa·s (10⁻³ Pa·s), alternatively 3 to 1,000 mPa·s (10⁻³ Pa·s), or alternatively 10 to 500 mPa·s (10⁻³ Pa·s).

The amount of SiH units present in the organohydrogensiloxane may vary, providing there are at least two SiH units per organohydrogensiloxane molecule. The amount of SiH units present in the organohydrogensiloxane is expressed herein as % SiH which is the weight percent of hydrogen in the organohydrogensiloxane. Typically, the % SiH varies from 0.01 to 10%, alternatively from 0.1 to 5%, or alternatively from 0.5 to 2%.

The organohydrogensiloxane may comprise the average formula;

(R³₃SiO_{1/2})ₐ(R⁴₂SiO_{2/2})_{b}(R⁴HSiO_{2/2})_{c} wherein
R³ is hydrogen or R⁴,
R⁴ is a monovalent hydrocarbon group having from 1 to 10 carbon atoms,
a≧2,
b≧0, alternatively b=1 to 500, alternatively b=1 to 200,
c≧2, alternatively c=2 to 200, alternatively c=2 to 100.

R⁴ may be a substituted or unsubstituted aliphatic or aromatic hydrocarbyl. Monovalent unsubstituted aliphatic hydrocarbyls are exemplified by, but not limited to alkyl groups such as methyl, ethyl, propyl, pentyl, octyl, undecyl, and octadecyl and cycloalkyl groups such as cyclohexyl. Monovalent substituted aliphatic hydrocarbyls are exemplified by, but not limited to halogenated alkyl groups such as chloromethyl, 3-chloropropyl, and 3,3,3-trifluoropropyl. The aromatic hydrocarbon group is exemplified by, but not limited to, phenyl, tolyl, xylyl, benzyl, styryl, and 2-phenylethyl.

The amounts of components b¹) and b²) used may vary, but typically the amounts of components b¹) and b²) are selected so as to provide a molar ratio of the alkenyl groups to SiH in the composition that is greater than 1.

Component b³) is a hydrosilylation catalyst. The hydrosilylation catalyst may be any suitable Group VIII metal based catalyst selected from a platinum, rhodium, iridium, palladium or ruthenium. Group VIII group metal containing catalysts useful to catalyze curing of the present compositions can be any of those known to catalyze reactions of silicon bonded hydrogen atoms with silicon bonded unsaturated hydrocarbon groups. The typical Group VIII metal for use as a catalyst to effect cure of the present compositions by hydrosilylation is a platinum based catalyst. Some typical platinum based hydrosilylation catalysts for curing the present composition are platinum metal, platinum compounds and platinum complexes. Suitable platinum catalysts are described in U.S. Pat. No. 2,823,218 (commonly referred to as “Speier's catalyst) and U.S. Pat. No. 3,923,705. The platinum catalyst may be “Karstedt's catalyst”, which is described in Karstedt's U.S. Pat. No. 3,715,334 and U.S. Pat. No. 3,814,730. Karstedt's catalyst is a platinum divinyl tetramethyl disiloxane complex typically containing about one-weight percent of platinum in a solvent such as toluene. Alternatively the platinum catalyst may be a reaction product of chloroplatinic acid and an organosilicon compound containing terminal aliphatic unsaturation, as described in U.S. Pat. No. 3,419,593. Alternatively, the hydrosilylation catalyst is a neutralized complex of platinum chloride and divinyl tetramethyl disiloxane, as described in U.S. Pat. No. 5,175,325.

The amounts of catalyst b³) used may vary, but typically an amount is used to effect the hydrosilylation reaction. When the catalyst is a Pt compound, typically a sufficient amount of the compound is added to provide 2 to 500 ppm of Pt in the silicone composition. Additional components may be added to the hydrosilylation reaction. For example, heptamethyltrisiloxysilane may be added as an endblocker to control molecular weight of the organopolysiloxane product.

When the silicone composition contains organopolysiloxanes components that can react via condensation, the silicone component comprises an organopolysiloxane having at least two siloxy units with a substituent capable of reacting via condensation. Suitable substitutes on the siloxy units of the organopolysiloxanes include silanol, alkoxy, acetoxy, oxime functional groups. In this embodiment, the silicone composition will further contain a catalyst known in the art for enhancing condensation cure of organopolysiloxanes such as a tin or titanium catalyst. In a further embodiment, the organopolysiloxane is a silanol endblocked polydimethylsiloxane having a kinematic viscosity that may range from 1 to 100,000 mm²/s (10⁻⁶ m²/s) at 25° C., or alternatively from 1 to 10,000 mm²/s (10⁻⁶ m²/s) at 25° C.

The silicone composition may contain organopolysiloxanes having at least one siloxy unit substituted with an organofunctional group. The organofunctional organopolysiloxanes useful in the present process are characterized by having at least one of the R groups in the formula RₙSiO_{(4-n)/2} be an organofunctional group. Representative non-limiting organofunctional groups include amino, amido, epoxy, mercapto, polyether (polyoxyalkylene) groups, and any mixture thereof. Further examples of organofunctional organopolysiloxanes include those having alkoxylated groups; hydroxyl groups such as the polyorganosiloxanes containing a hydroxyalkyl function, as described in EP1081272, U.S. Pat. No. 6,171,515 and U.S. Pat. No. 6,136,215; Bis-Hydroxy/Methoxy Amodimethicone; amino-acid functional siloxanes obtained by reacting an amino acid derivative selected from the group of an N-acyl amino acid and an N-aroyl amino acid with an amino functional siloxane, further described in WO2007/141565; quaternary ammonium functional silicones, described in U.S. Pat. No. 6,482,969 and U.S. Pat. No. 6,607,717, such as Silicone Quaternium-16 (CTFA designation); hydrocarbyl functional organopolysiloxanes comprising a siloxy unit of the formula R⁵R′ᵢSiO_{(3-i)/2} wherein R′ is any monovalent hydrocarbon group, but typically is an alkyl, cycloalkyl, alkenyl, alkaryl, aralkyl, or aryl group containing 1-20 carbon atoms, R⁵ is a hydrocarbyl group having the formula —R⁶OCH₂CH₂OH, wherein R⁶ is a divalent hydrocarbon group containing 2 to 6 carbon atoms and i has a value of from zero to 2, such as described in U.S. Pat. No. 2,823,218, U.S. Pat. No. 5,486,566, U.S. Pat. No. 6,060,044 and US20020524 (CTFA Bis-Hydroxyethoxypropyl Dimethicone); siloxane-based polyamide such as described in U.S. Pat. No. 6,051,216; silicone polyether-amide block copolymers such as described in US2008/0045687.

The organofunctional group may be present on any siloxy unit having an R substituent, that is, they may be present on any (R₃SiO_{0.5}), (R₂SiO), or (RSiO_{1.5}) unit.

The organofunctional group may be an amino-functional group. Amino-functional groups may be designated in the formulas herein as R^{N} and is illustrated by groups having the formula: —R⁸NHR⁹, —R⁸NR⁹₂, or —R⁸NHR⁸NHR⁹, wherein each R⁸ is independently a divalent hydrocarbon group having at least 2 carbon atoms, and R⁹ is hydrogen or an alkyl group. Each R⁸ is typically an alkylene group having from 2 to 20 carbon atoms. Some examples of suitable amino-functional hydrocarbon groups are; —CH₂CH₂NH₂, —CH₂CH₂CH₂NH₂, —CH₂CHCH₃NH, —CH₂CH₂CH₂CH₂NH₂, —CH₂CH₂CH₂CH₂CH₂NH₂, —CH₂CH₂CH₂CH₂CH₂CH₂NH₂, —CH₂CH₂NHCH₃, —CH₂CH₂CH₂NHCH₃, —CH₂(CH₃)CHCH₂NHCH₃, —CH₂CH₂CH₂CH₂NHCH₃, —CH₂CH₂NHCH₂CH₂NH₂, —CH₂CH₂CH₂NHCH₂CH₂NH₂, —CH₂CH₂CH₂NHCH₂CH₂CH₂NH₂, —CH₂CH₂CH₂CH₂NHCH₂CH₂CH₂CH₂NH₂, —CH₂CH₂NHCH₂CH₂NHCH₃, —CH₂CH₂CH₂NHCH₂CH₂CH₂NHCH₃, —CH₂CH₂CH₂CH₂NHCH₂CH₂CH₂CH₂NHCH₃, and —CH₂CH₂NHCH₂CH₂NHCH₂CH₂CH₂CH₃.

Examples of silicone resins include trimethylsilylsilicate (MQ resin), silsesquioxane resins (T resin), MQ-T resins, silsesquioxane resin waxes.

A trimethylsilylsilicate (MQ resin) may comprise ≧80 mole % of siloxy units selected from (R¹⁰₃SiO_{1/2})ₐ and (SiO_{4/2})_{d} units, where R¹⁰ is an alkyl group having from 1 to 8 carbon atoms, an aryl group, a carbinol group, or an amino group, with the proviso that ≧95 mole % of the R¹⁰ groups are alkyl groups, a and d>0, and the ratio of a/d=0.5 to 1.5.

MQ resins may contain D and T units, providing that ≧80 mole %, alternatively ≧90 mole % of the total siloxane units are M and Q units. The MQ resins may also contain hydroxy groups. Typically, the MQ resins have a total weight % hydroxy content of 2 to 10 weight %, alternatively 2 to 5 weight %. The MQ resins can also be further “capped” wherein residual hydroxy groups are reacted further with M groups.

A silsesquioxane resins (T resin) may comprise ≧30 mole % of R¹⁰SiO_{3/2} units, where R¹⁰ is as defined above. When ≧40 mole % of the R¹⁰ groups are propyl, the T resin may be named a propyl silsesquioxane resin.

T resins may contain M, D, and Q units, providing that ≧30 mole %, alternatively ≧80 mole %, alternatively ≧90 mole % of the total siloxane units are T units. The T resins may also contain hydroxy and/or alkoxy groups. Typically, the T resins have a total weight % hydroxy content of 2 to 10 weight % and a total weight % alkoxy content >20 weight %; alternatively 6 to 8 weight % hydroxy content and ≦10 weight % alkoxy content.

MQ and T organopolysiloxane resins may be used alone or combined together.

A MQ-T resin may have the formula (R¹¹₃SiO_{1/2})ₐ(R¹²₂SiO_{2/2})_{b}(R¹³SiO_{3/2})_{c}(SiO_{4/2})_{d} with R¹¹, R¹² and R¹³ independently represent an alkyl group containing from 1 to 8 carbon atoms, an aryl group, a carbinol group or an amino group, where 0.05≦a≦0.5; 0≦b≦0.3; c>0; 0.05≦d≦0.6, and a+b+c+d=1, with the proviso that ≧40 mole % of the R¹³ groups in the siloxane resin are propyl. Representatives of such MQ-T resins are taught in WO2005/075542, incorporated herein by reference.

A silsesquioxane resin wax may comprise at least 40 mole % of siloxy units having the formula (R¹⁰₂R¹⁴SiO_{1/2})ₓ(R¹⁵SiO_{3/2})_{y}, where x and y have a value of 0.05 to 0.95, R¹⁰ is as described above, R¹⁴ is a monovalent hydrocarbon having 9-40 carbon atoms, and R¹⁵ is a monovalent hydrocarbon group having 1 to 8 carbon atoms or an aryl group. R¹⁴ and the ratio of y/x are selected such that the silsesquioxane resin wax has a melting point of ≧about 30° C. Representatives of such silsesquioxane resin waxes are taught in U.S. Pat. No. 7,482,419, incorporated herein by reference.

Examples of silicone waxes include C30-45 alkyl methicone and C30-45 olefin (MP>60° C.), Bis-PEG-18 methyl ethyl dimethyl silane, stearyl dimethicone.

Silicone elastomers are a type of tri-dimensional crosslinked silicone polymers. Examples of silicone elastomers include those obtained from the crosslinking hydrosilylation reaction of an organohydrogenpolysiloxane with another polysiloxane containing an unsaturated hydrocarbon substituent, such as a vinyl functional polysiloxane, or by crosslinking an organohydrogenpolysiloxane with a hydrocarbon diene or with a terminally unsaturated polyoxyalkylene. Representative examples of such silicone elastomers are taught in U.S. Pat. No. 5,880,210 and U.S. Pat. No. 5,760,116. In some instances, organofunctional groups have been grafted onto the silicone organic elastomer backbone, such as alkyls, polyether, amines. Representative examples of such organofunctional silicone elastomers are taught in U.S. Pat. No. 5,811,487, U.S. Pat. No. 5,880,210, U.S. Pat. No. 6,200,581, U.S. Pat. No. 5,236,986, U.S. Pat. No. 6,331,604, U.S. Pat. No. 6,262,170, U.S. Pat. No. 6,531,540, and U.S. Pat. No. 6,365,670, WO2004/104013 and WO2004/103326.

Examples of saccharide-siloxane polymer include the reaction product of a functionalized organosiloxane polymer and at least one hydroxy-functional saccharide component comprising 5 to 12 carbon atoms, in such a way that the organosiloxane component is covalently linked via a linking group to the saccharide component. Saccharide-siloxane polymers may be linear or branched. Further examples of saccharide-siloxane polymers are known by the skilled in the art, and described in US20080199417, US20100105582, WO2012027073, WO2012027143 incorporated herein by reference.

Examples of vinyl polymer grafted with a carbosiloxane dendrimers include the reaction product of a vinyl polymer with at least one carbosiloxane dendrimer-based unit. The term “carbosiloxane dendrimer structure” designates a structure with branched groups of high molecular masses with high regularity in the radial direction starting from the simple backbone. Such carbosiloxane dendrimer structures are described in the form of a highly branched siloxane-silalkylene copolymer in the laid-open Japanese patent application Kokai 9-171 154. Other vinyl polymers grafted with a carbosiloxane dendrimer are known by the skilled in the art, and described in EP0963751 incorporated herein by reference.

### The Second Dispersed Phase of Bi-Modal Emulsion (E)

The bi-modal water continuous emulsions have a second dispersed phase containing a silicone that is provided from a water continuous silicone emulsion containing at least one surfactant (designated herein as component (B)). The water continuous silicone emulsion containing at least one surfactant (B) may be a single water continuous silicone emulsion, or a combination of water continuous silicone emulsions.

The water continuous silicone emulsion(s) (B) useful in the present bi-modal emulsions contains at least one surfactant. The surfactant may vary, but typically is chosen from those surfactants that enhance the formation of water continuous emulsions. The surfactant may be an anionic surfactant, cationic surfactant, nonionic surfactant, amphoteric surfactant, or a mixture of any of these surfactants.

The silicone in the water continuous silicone emulsion containing at least one surfactant (B) may be any of those listed above as hydrophobic oil (A), and mixtures thereof.

Examples of anionic surfactants include alkali metal, amine, or ammonium salts of higher fatty acids, alkylaryl sulphonates such as sodium dodecyl benzene sulfonate, long chain fatty alcohol sulfates, olefin sulfates and olefin sulfonates, sulfated monoglycerides, sulfated esters, sulfonated ethoxylated alcohols, sulfosuccinates, alkane sulfonates, phosphate esters, alkyl isethionates, alkyl taurates, alkyl sarcosinates, and mixtures thereof.

Examples of cationic surfactants include alkylamine salts, quaternary ammonium salts, sulphonium salts, and phosphonium salts. Examples of suitable nonionic surfactants include condensates of ethylene oxide with long chain fatty alcohols or fatty acids such as a C₁₂₋₁₆ alcohol, condensates of ethylene oxide with an amine or an amide, condensation products of ethylene and propylene oxide, esters of glycerol, sucrose, sorbitol, fatty acid alkylol amides, sucrose esters, fluoro-surfactants, fatty amine oxides, and mixtures thereof.

Examples of amphoteric surfactants include imidazoline compounds, alkylaminoacid salts, betaines, and mixtures thereof.

Examples of nonionic surfactants include polyoxyethylene fatty alcohols such as polyoxyethylene (23) lauryl ether, polyoxyethylene (4) lauryl ether; ethoxylated alcohols such as ethoxylated trimethylnonanol, C₁₂-C₁₄ secondary alcohol ethoxylates, ethoxylated, C10-Guerbet alcohol, ethoxylated, iso-C13 alcohol; poly(oxyethylene)-poly(oxypropylene)-poly(oxyethylene) tri-block copolymer (also referred to as poloxamers); tetrafunctional poly(oxyethylene)-poly(oxypropylene) block copolymer derived from the sequential addition of propylene oxide and ethylene oxide to ethylene diamine (also referred to as poloxamines), silicone polyethers, and mixtures thereof.

When mixtures containing nonionic surfactants are used, one nonionic surfactant may have a low Hydrophile-Lipophile Balance (HLB) and the other nonionic surfactant may have a high HLB, such that the two nonionic surfactants have a combined HLB of 11-15, alternatively a combined HLB of 12.5-14.5.

The water continuous silicone emulsion (B) may be selected from those considered in the art to be a “macro” or “micro” emulsion. In other words, the average particle size of the water continuous emulsion may vary from 0.001 to 1000 μm, alternatively from 0.01 to 20 μm, or alternatively from 0.02 to 10 μm.

The water continuous silicone emulsion (B) is a microemulsion having an average particle size of less than 100 nm.

The water continuous silicone emulsion (B) may be considered an “emulsion polymer”, in other words, an emulsion formed by emulsion polymerization techniques. Examples of suitable silicone emulsions produced by emulsion polymerization techniques are taught in U.S. Pat. No. 2,891,920, U.S. Pat. No. 3,294,725, U.S. Pat. No. 5,661,215, U.S. Pat. No. 5,817,714, and U.S. Pat. No. 6,316,541, which are incorporated herein by reference.

The water continuous silicone emulsion (B) may be a mechanical emulsion. As used herein, mechanical emulsions refer to those emulsion in the art produced by using mechanical energy (such as from high shearing forces). Examples of silicone emulsions produced by mechanical techniques are taught in U.S. Pat. No. 6,395,790, which is incorporated herein by reference.

The water continuous silicone emulsion may be prepared using suspension polymerization techniques. Examples of silicone emulsions produced by suspension polymerization techniques are taught in U.S. Pat. No. 4,618,645, U.S. Pat. No. 6,248,855, and U.S. Pat. No. 6,395,790.

The present bi-modal water continuous emulsion (E) may be prepared by a process comprising:
I) forming a mixture comprising;
   A) 100 parts by weight of a hydrophobic oil,
   B) 1 to 1000 part by weight of a water continuous silicone emulsion having at least one surfactant,
II) admixing additional quantities of the water continuous emulsion and/or water to the mixture from step I) to form a bi-modal emulsion.

Component (A) in step I) of the above process may be any hydrophobic oil as described above as component (A) in the first dispersed phase.

Component (B) in step I) of the above process may be any water continuous silicone emulsion as described above as component (B) in the second dispersed phase.

Mixing in step I) can be accomplished by any method known in the art to effect mixing of high viscosity materials. The mixing may occur either as a batch, semi-continuous, or continuous process. Mixing may occur, for example using, batch mixing equipments with medium/low shear include change-can mixers, double-planetary mixers, conical-screw mixers, ribbon blenders, double-arm or sigma-blade mixers; batch equipments with high-shear and high-speed dispersers include those made by Charles Ross & Sons (NY), Hockmeyer Equipment Corp. (NJ); batch equipments with high shear actions include Banbury-type (CW Brabender Instruments Inc., NJ) and Henschel type (Henschel mixers America, TX). Illustrative examples of continuous mixers/compounders include extruders single-screw, twin-screw, and multi-screw extruders, co-rotating extruders, such as those manufactured by Krupp Werner & Pfleiderer Corp (Ramsey, N.J.), and Leistritz (NJ); twin-screw counter-rotating extruders, two-stage extruders, twin-rotor continuous mixers, dynamic or static mixers or combinations of these equipments.

The temperature and pressure at which the mixing of step I) occurs is not critical, but generally is conducted at ambient temperature and pressures. Typically, the temperature of the mixture will increase during the mixing process due to the mechanical energy associated when shearing such high viscosity materials.

Typically 1 to 1000 parts by weight of the water continuous emulsion are mixed for every 100 parts by weight of component (A) in the step I) mixture, alternatively from 5 to 500 parts per 100 parts by weight of component (A) in the step I) mixture, or alternatively from 5 to 100 parts per 100 parts by weight of component (A) the step I) mixture.

Step I) may involve forming a mixture consisting essentially of;
A) 100 parts by weight of a hydrophobic oil,
B) 1 to 1000 parts by weight of a water continuous emulsion having at least one surfactant.
   In this embodiment, the mixture formed in step I) is “essentially free” from any other surfactant compounds or components other than components (A) and (B). As used herein, “essentially free” means no other surfactant compounds are added to the mixture formed in step I), other than the surfactant(s) present in (B) the water continuous emulsion.

Step II) of the process involves admixing additional quantities of the water continuous emulsion and/or water to the mixture from step I) to form a bi-modal emulsion.

The amount of the additional quantities of the water continuous emulsion and/or water used in step II) may vary depending on the selection of components (A) and (B). Typically the amount of additional water continuous emulsion and/or water admixed in step II) of the present process may vary from 1 to 1000 parts by weight of the step I) mixture, alternatively from 5 to 500 parts per 100 parts by weight, or alternatively from 5 to 100 parts per 100 parts by weight.

In step II) of the present process, additional quantities of the water continuous emulsion may be used alone, or alternatively be combined with varying quantities of water. Alternatively, additional quantities of water may be added alone without any additional quantities of the water continuous emulsion. The selection of using additional quantities of the water continuous emulsion alone, in combination with varying amounts of water, or water alone will depend on the initial selection of the water continuous emulsion and the desired physical properties of the resulting bi-modal emulsion. For example, high solids bi-modal emulsions may be prepared with only the addition of the water continuous emulsion. Conversely, low solids bi-modal emulsions may require the addition of water as well.

The water continuous emulsion and/or water is added to the mixture from step I) at such a rate, with additional mixing, so as to form an emulsion of the mixture of step I). The water continuous emulsion added to the mixture from step I) may be done in incremental portions, whereby each incremental portion comprises less than 50 weight % of the mixture from step I), alternatively 25 weight % of the mixture from step I), and each incremental portion of water continuous emulsion is added successively to the previous after the dispersion of the previous incremental portion of water continuous emulsion, wherein sufficient incremental portions of water continuous emulsion are added to form the bi-modal emulsion.

The number of incremental portions of the water continuous emulsion and/or water added to the mixture from step I) may vary, but typically at least 2, alternatively, at least 3 incremental portions are added.

Mixing in step II) can be accomplished by any method known in the art to effect mixing of high viscosity materials and/or effect the formation of an emulsion. The mixing may occur either as a batch, semi-continuous, or continuous process. Any of the mixing methods as described for step I), may be used to effect mixing in step II). Alternatively, mixing in step II) may also occur via those techniques known in the art to provide high shear mixing to effect formation of emulsions. Representative of such high shear mixing techniques include; high speed stirrers, homogenizers, Sonolators®, Microfluidizers®, Ross mixers, Eppenbach colloid mills, Flacktek Speedmixers®, and other similar shear devices.

Optionally, the emulsion formed in step II) may be further sheared according to an optional step III) to reduce particle size and/or improve long term storage stability. The shearing may occur by any of the mixing techniques discussed above.

The bi-modal water continuous emulsions prepared by the present process may be characterized by their bi-modal particle size distribution. The particle size may be determined by laser diffraction of the emulsion. Suitable laser diffraction techniques are well known in the art. The particle size is obtained from a particle size distribution (PSD). The PSD can be determined on a volume, surface, length basis. The volume particle size is equal to the diameter of the sphere that has the same volume as a given particle. The term Dv, as used herein, represents the average volume particle size of the dispersed particles. Dv 50 is the particle size measured in volume corresponding to 50% of the cumulative particle population. In other words if Dv 50=10 μm, 50% of the particle have an average volume particle size below 10 μm and 50% of the particle have a volume average particle size above 10 μm. Dv 90 is the particle size measured in volume corresponding to 90% of the cumulative particle population. Mode 1 is the median of the distribution of one of the dispersed phase particle populations within a bimodal particle size distribution and Mode 2 is the median of the other.

In some instances, it may be necessary to conduct two separate evaluations of particle size, especially when the particle sizes distributions of the resulting bi-modal emulsions exhibit a wide variation in size. In these instances a Malvern-Mastersizer® 2000 may be used to obtain particle size distributions in the range 0.5 to 1000 μm, while a Microtrac-Nanotrac® may be used to measure particle size distributions in the ranges less than 0.5 μm.

The average volume particle size of the dispersed particles in the oil/water emulsions ranges of from 0.001 μm to 1000 μm; or of from 0.01 μm to 20 μm; or of from 0.02 μm to 10 μm.

Alternatively, the average volume particle size of each of the unique dispersed phases (that is the first dispersed phase, and the second dispersed phase), may be reported. The average volume particle size of the first dispersed particles in the oil/water emulsions ranges of from 0.1 μm to 500 μm; or of from 0.1 μm to 100 μm; or of from 0.2 μm to 30 μm. The average volume particle size of the second dispersed particles in the oil/water emulsions ranges of from 0.1 μm to 500 μm; or of from 0.1 μm to 100 μm; or of from 0.2 μm to 30 μm.

While not wishing to be bound by any theory, it is believed particle size distribution of the first dispersed phase results from the emulsification of the hydrophobic oil, while particle size distribution of the second dispersed phase results from the particles originating from the water continuous emulsion used in the present process. However, there may be certain instances where the two overlap sufficiently that a bi-modal distribution is not observable using the particle size determination techniques described above.

The bimodal particle size distribution may also be observed using optical microscopy techniques.

In some instances, the bi-modal emulsions may be considered as a “high solids” emulsion, wherein the bi-modal emulsion contains at least 75% by weight of components (A) and (B), alternatively the bi-modal emulsion contains at least 80% by weight of components (A) and (B), alternatively the bi-modal emulsion contains at least 85% by weight of components (A) and (B), alternatively the bi-modal emulsion contains at least 90% by weight of components (A) and (B).

The “high solids” bi-modal emulsion may remain pourable. Thus, the bi-modal emulsions may have a viscosity less than 600,000 mPa·s (10⁻³ Pa·s), alternatively less than 200,000 mPa·s (10⁻³ Pa·s), or alternatively less than 100,000 mPa·s (10⁻³ Pa·s), as measured at 25° C.

The total surfactant concentration in the bi-modal emulsion is less than 4.0 weight %, alternatively less than 1.0 weight %, or alternatively less than 0.2 weight %. The bi-modal silicone emulsions produced by the present process contains less than 1.0 weight % cyclosiloxanes, alternatively contains less than 0.5 weight % cyclosiloxanes, alternatively contains less than 0.1 weight % cyclosiloxanes.

The bi-modal water continuous emulsion (E) is present in a cosmetic composition in conjunction with a cosmetic ingredient (C), optionally in a cosmetically acceptable medium.
Cosmetic compositions include those compositions which are intended to be placed in contact with the external parts of the human body (skin (epidermis), hair system, nails, mucosa, etc., also referred to as “keratinous substrates”) or with the teeth and the mucous membranes of the oral cavity with a view exclusively or mainly to cleaning them, perfuming them, changing their appearance, protecting them, keeping them in good condition or correcting body odours. In some instances, cosmetic compositions may also include health care compositions.
Cosmetic applications, and in some instances health care applications, include skin care, hair care, or nail care applications.

Cosmetic ingredients are those ingredients known to be used in cosmetic application. A wide review of such ingredients may be found in the CTFA cosmetic ingredient handbook.
Cosmetically acceptable medium include water, solvents, diluents, or mixtures and emulsions thereof.

Cosmetic ingredients include emollients, waxes, moisturizers, surface active materials such as surfactants or detergents or emulsifiers, thickeners, water phase stabilizing agents, pH controlling agents, preservatives and cosmetic biocides, sebum absorbants or sebum control agents, vegetable or botanical extracts, vitamins, proteins or amino-acids and their derivatives, pigments, colorants, fillers, silicone conditioning agents, cationic conditioning agents, hydrophobic conditioning agents, UV absorbers, sunscreen agents, antidandruff agents, antiperspirant agents, deodorant agents, skin protectants, hair dyes, nail care ingredients, fragrances or perfume, antioxidants, oxidizing agents, reducing agents, propellant gases, film formers and mixtures thereof.

Additional ingredients that may be used in the cosmetic compositions include fatty alcohols, colour care additives, anticellulites, pearlising agents, chelating agents, styling agents, ceramides, suspending agents and others.

Health care ingredients include antiacne agents, antibacterial agents, antifungal agents, therapeutic active agents, external analgesics, skin bleaching agents, anti-cancer agents, diuretics, agents for treating gastric and duodenal ulcers, proteolytic enzymes, antihistamine or H1 histamine blockers, sedatives, bronchodilators, diluents.

Additional ingredients that may be used in the health care compositions include antibiotic, antiseptic, antibacterial, anti-inflammatory, astringents, hormones, smoking cessation compositions, cardiovascular, antiarrythmic, alpha-I blocker, beta blocker, ACE inhibitor, antiaggregant, non-steroidal anti-inflammatory agents such as diclofenac, antipsoriasis agents such as clobetasol propionate, antidermatitis agents, tranquillizer, anticonvulsant, anticoagulant agents, healing factors, cell growth nutrients, peptides, corticosteroidal drugs, antipruritic agents and others.

Cosmetic ingredients may be used in health care compositions, such as waxes, and others; and health care ingredients may be used in cosmetic compositions such as anti-acne agents, and others.

Examples of emollients include volatile or non-volatile silicone oils; silicone resins such as polypropylsilsesquioxane and phenyl trimethicone; silicone elastomers such as dimethicone crosspolymer; alkylmethylsiloxanes such as C30-45 Alkyl Methicone; volatile or non-volatile hydrocarbon compounds, such as squalene, paraffin oils, petrolatum oils and naphthalene oils; hydrogenated or partially hydrogenated polyisobutene; isoeicosane; squalane; isoparaffin; isododecane; isodecane or isohexa-decane; branched C8-C16 esters; isohexyl neopentanoate; ester oils such as isononyl isononanoate, cetostearyl octanoate, isopropyl myristate, palmitate derivatives, stearates derivatives, isostearyl isostearate and the heptanoates, octanoates, decanoates or ricinoleates of alcohols or of polyalcohols, or mixtures thereof; hydrocarbon oils of plant origin, such as wheatgerm, sunflower, grapeseed, castor, shea, avocado, olive, soybean, sweet almond, palm, rapeseed, cotton seed, hazelnut, macadamia, jojoba, blackcurrant, evening primrose; or triglycerides of caprylic/capric acids; higher fatty acids, such as oleic acid, linoleic acid or linolenic acid, and mixtures thereof.

Example of waxes include hydrocarbon waxes such as beeswax, lanolin wax, rice wax, carnauba wax, candelilla wax, microcrystalline waxes, paraffins, ozokerite, polyethylene waxes, synthetic wax, ceresin, lanolin, lanolin derivatives, cocoa butter, shellac wax, bran wax, capok wax, sugar cane wax, montan wax, whale wax, bayberry wax, silicone waxes (e.g. polymethylsiloxane alkyls, alkoxys and/or esters, C30-45 alkyldimethylsilyl polypropylsilsesquioxane), and mixtures thereof.

Examples of moisturizers include lower molecular weight aliphatic diols such as propylene glycol and butylene glycol; polyols such as glycerine and sorbitol; and polyoxyethylene polymers such as polyethylene glycol 200; hyaluronic acid and its derivatives, and mixtures thereof.

Examples of surface active materials may be anionic, cationic or non ionic, and include organomodified silicones such as dimethicone copolyol; oxyethylenated and/or oxypropylenated ethers of glycerol; oxyethylenated and/or oxypropylenated ethers of fatty alcohols such as ceteareth-30, C12-15 pareth-7; fatty acid esters of polyethylene glycol such as PEG-50 stearate, PEG-40 monostearate; saccharide esters and ethers, such as sucrose stearate, sucrose cocoate and sorbitan stearate, and mixtures thereof; phosphoric esters and salts thereof, such as DEA oleth-10 phosphate; sulphosuccinates such as disodium PEG-5 citrate lauryl sulphosuccinate and disodium ricinoleamido MEA sulphosuccinate; alkyl ether sulphates, such as sodium lauryl ether sulphate; isethionates; betaine derivatives; and mixtures thereof.

Further examples of nonionic surfactants include polyoxyethylene alkyl ethers, polyoxyethylene alkylphenol ethers, polyoxyethylene lauryl ethers, polyoxyethylene sorbitan monoleates, polyoxyethylene alkyl esters, polyoxyethylene sorbitan alkyl esters, polyethylene glycol, polypropylene glycol, diethylene glycol, ethoxylated trimethylnonanols, polyoxyalkylene-substituted silicones (rake or ABn types), silicone alkanolamides, silicone esters, silicone glycosides, and mixtures thereof.

Nonionic surfactants include dimethicone copolyols, fatty acid esters of polyols, for instance sorbitol or glyceryl mono-, di-, tri- or sesquioleates or stearates, glyceryl or polyethylene glycol laurates; fatty acid esters of polyethylene glycol (polyethylene glycol monostearate or monolaurate); polyoxyethylenated fatty acid esters (stearate or oleate) of sorbitol; polyoxyethylenated alkyl (lauryl, cetyl, stearyl or octyl)ethers.

Anionic surfactants include carboxylates (sodium 2-(2-hydroxyalkyloxy)acetate)), amino acid derivatives (N-acylglutamates, N-acylgly-cinates or acylsarcosinates), alkyl sulfates, alkyl ether sulfates and oxyethylenated derivatives thereof, sulfonates, isethionates and N-acylisethionates, taurates and N-acyl N-methyltaurates, sulfosuccinates, alkylsulfoacetates, phosphates and alkyl phosphates, polypeptides, anionic derivatives of alkyl polyglycoside (acyl-D-galactoside uronate), and fatty acid soaps, and mixtures thereof.

Amphoteric and zwitterionic surfactants include betaines, N-alkylamidobetaines and derivatives thereof, proteins and derivatives thereof, glycine derivatives, sultaines, alkyl polyaminocarboxylates and alkylamphoacetates, and mixtures thereof.

Examples of thickeners include acrylamide polymers and copolymers, acrylate copolymers and salts thereof (such as sodium polyacrylate), xanthan gum and derivatives, cellulose gum and cellulose derivatives (such as methylcellulose, methylhydroxypropylcellulose, hydroxypropylcellulose, polypropylhydroxyethylcellulose), starch and starch derivatives (such as hydroxyethylamylose and starch amylase), polyoxyethylene, carbomer, hectorite and hectorite derivatives, sodium alginate, arabic gum, cassia gum, guar gum and guar gum derivatives, cocamide derivatives, alkyl alcohols, gelatin, PEG-derivatives, saccharides (such as fructose, glucose) and saccharides derivatives (such as PEG-120 methyl glucose diolate), and mixtures thereof.

Examples of water phase stabilizing agents include electrolytes (e.g. alkali metal salts and alkaline earth salts, especially the chloride, borate, citrate, and sulfate salts of sodium, potassium, calcium and magnesium, as well as aluminum chlorohydrate, and polyelectrolytes, especially hyaluronic acid and sodium hyaluronate), polyols (glycerine, propylene glycol, butylene glycol, and sorbitol), alcohols such as ethyl alcohol, and hydrocolloids, and mixtures thereof.

Examples of pH controlling agents include any water soluble acid such as a carboxylic acid or a mineral acid such as hydrochloric acid, sulphuric acid, and phosphoric acid, monocarboxylic acid such as acetic acid and lactic acid, and polycarboxylic acids such as succinic acid, adipic acid, citric acid, and mixtures thereof.

Example of preservatives and cosmetic biocides include paraben derivatives, hydantoin derivatives, chlorhexidine and its derivatives, imidazolidinyl urea, phenoxyethanol, silver derivatives, salicylate derivatives, triclosan, ciclopirox olamine, hexamidine, oxyquinoline and its derivatives, PVP-iodine, zinc salts and derivatives such as zinc pyrithione, and mixtures thereof.

Examples of sebum absorbants or sebum control agents include silica silylate, silica dimethyl silylate, dimethicone/vinyl dimethicone crosspolymer, polymethyl methacrylate, cross-linked methylmethacrylate, aluminum starch octenylsuccinate, and mixtures thereof.

Examples of vegetable or botanical extracts are derived from plants (herbs, roots, flowers, fruits, or seeds) in oil or water soluble form, such as coconut, green tea, white tea, black tea, horsetail, ginkgo biloba, sunflower, wheat germ, seaweed, olive, grape, pomegranate, aloe, apricot kernel, apricot, carrot, tomato, tobacco, bean, potato, actzuki bean, catechu, orange, cucumber, avocado, watermelon, banana, lemon or palm. Examples of herbal extracts include dill, horseradish, oats, neem, beet, broccoli, tea, pumpkin, soybean, barley, walnut, flax, ginseng, poppy, avocado, pea, sesame, and mixtures thereof.

Examples of vitamins include a variety of different organic compounds such as alcohols, acids, sterols, and quinones. They may be classified into two solubility groups: lipid-soluble vitamins and water-soluble vitamins. Lipid-soluble vitamins that have utility in personal care formulations include retinol (vitamin A), ergocalciferol (vitamin D2), cholecalciferol (vitamin D3), phytonadione (vitamin K1), and tocopherol (vitamin E). Water-soluble vitamins that have utility in personal care formulations include ascorbic acid (vitamin C), thiamin (vitamin B1) niacin (nicotinic acid), niacinamide (vitamin B3), riboflavin (vitamin B2), pantothenic acid (vitamin B5), biotin, folic acid, pyridoxine (vitamin B6), and cyanocobalamin (vitamin B12). Additional examples of vitamins include derivatives of vitamins such as retinyl palmitate (vitamin A palmitate), retinyl acetate (vitamin A acetate), retinyl linoleate (vitamin A linoleate), and retinyl propionate (vitamin A propionate), tocopheryl acetate (vitamin E acetate), tocopheryl linoleate (vitamin E linoleate), tocopheryl succinate (vitamin E succinate), tocophereth-5, tocophereth-10, tocophereth-12, tocophereth-18, tocophereth-50 (ethoxylated vitamin E derivatives), PPG-2 tocophereth-5, PPG-5 tocophereth-2, PPG-10 tocophereth-30, PPG-20 tocophereth-50, PPG-30 tocophereth-70, PPG-70 tocophereth-100 (propoxylated and ethoxylated vitamin E derivatives), sodium tocopheryl phosphate, ascorbyl palmitate, ascorbyl dipalmitate, ascorbyl glucoside, ascorbyl tetraisopalmitate, tetrahexadecyl ascorbate, ascorbyl tocopheryl maleate, potassium ascorbyl tocopheryl phosphate, tocopheryl nicotinate, and mixtures thereof.

Examples of proteins or amino-acids and their derivatives include those extracted from wheat, soy, rice, corn, keratin, elastin or silk. Proteins may be in the hydrolyzed form and they may also be quaternized, such as hydrolyzed elastin, hydrolyzed wheat powder, hydrolyzed silk. Examples of protein include enzymes such as hydrolases, cutinases, oxidases, transferases, reductases, hemicellulases, esterases, isomerases, pectinases, lactases, peroxidases, laccases, catalases, and mixtures thereof. Examples of hydrolases include proteases (bacterial, fungal, acid, neutral or alkaline), amylases (alpha or beta), lipases, mannanases, cellulases, collagenases, lisozymes, superoxide dismutase, catalase, and mixtures thereof.

Examples of pigments and colorants include surface treated or untreated iron oxides, surface treated or untreated titanium dioxide, surface treated or untreated mica, silver oxide, silicates, chromium oxides, carotenoids, carbon black, ultramarines, chlorophyllin derivatives and yellow ocher. Examples of organic pigments include aromatic types including azo, indigoid, triphenylmethane, anthraquinone, and xanthine dyes which are designated as D&C and FD&C blues, browns, greens, oranges, reds, yellows, etc, and mixtures thereof. Surface treatments include those treatments based on lecithin, silicone, silanes, fluoro compounds, and mixtures thereof.

Examples of fillers include talc, micas, kaolin, zinc or titanium oxides, calcium or magnesium carbonates, silica, silica silylate, titanium dioxide, glass or ceramic beads, polymethylmethacrylate beads, boron nitride, aluminum silicate, aluminum starch octenylsuccinate, bentonite, magnesium aluminum silicate, nylon, silk powder metal soaps derived from carboxylic acids having 8-22 carbon atoms, non-expanded synthetic polymer powders, expanded powders and powders from natural organic compounds, such as cereal starches, which may or may not be crosslinked, copolymer microspheres, polytrap, silicone resin microbeads, and mixtures thereof. The fillers may be surface treated to modify affinity or compatibility with remaining ingredients.

Examples of silicone conditioning agents include silicone oils such as dimethicone; silicone gums such as dimethiconol; silicone resins such as trimethylsiloxy silicate, polypropyl silsesquioxane; silicone elastomers; alkylmethylsiloxanes; organomodified silicone oils, such as amodimethicone, aminopropyl phenyl trimethicone, phenyl trimethicone, trimethyl pentaphenyl trisiloxane, silicone quaternium-16/glycidoxy dimethicone crosspolymer, silicone quaternium-16; saccharide functional siloxanes; carbinol functional siloxanes; silicone polyethers; siloxane copolymers (divinyldimethicone/dimethicone copolymer); acrylate or acrylic functional siloxanes; and mixtures or emulsions thereof.

Examples of cationic conditioning agents include guar derivatives such as hydroxypropyltrimethylammonium derivative of guar gum; cationic cellulose derivatives, cationic starch derivatives; quaternary nitrogen derivatives of cellulose ethers; homopolymers of dimethyldiallyl ammonium chloride; copolymers of acrylamide and dimethyldiallyl ammonium chloride; homopolymers or copolymers derived from acrylic acid or methacrylic acid which contain cationic nitrogen functional groups attached to the polymer by ester or amide linkages; polymeric quaternary ammonium salts of hydroxyethyl cellulose reacted with a fatty alkyl dimethyl ammonium substituted epoxide ; polycondensation products of N,N′-bis-(2,3-epoxypropyl)-piperazine or piperazine-bis-acrylamide and piperazine; and copolymers of vinylpyrrolidone and acrylic acid esters with quaternary nitrogen functionality. Specific materials include the various polyquats Polyquaternium-7, Polyquaternium-8, Polyquaternium-10, Polyquaternium-11, and Polyquaternium-23. Other categories of conditioners include cationic surfactants such as cetyl trimethylammonium chloride, cetyl trimethylammonium bromide, stearyltrimethylammonium chloride, and mixtures thereof. In some instances, the cationic conditioning agent is also hydrophobically modified, such as hydrophobically modified quatemized hydroxyethylcellulose polymers; cationic hydrophobically modified galactomannan ether; and mixtures thereof.

Examples of hydrophobic conditioning agents include guar derivatives; galactomannan gum derivatives; cellulose derivatives; and mixtures thereof.
UV absorbers and sunscreen agents include those which absorb ultraviolet light between about 290-320 nanometers (the UV-B region) and those which absorb ultraviolet light in the range of 320-400 nanometers (the UV-A region).

Some examples of sunscreen agents are aminobenzoic acid, cinoxate, diethanolamine methoxycinnamate, digalloyl trioleate, dioxybenzone, ethyl 4-[bis(Hydroxypropyl)]aminobenzoate, glyceryl aminobenzoate, homosalate, lawsone with dihydroxyacetone, menthyl anthranilate, octocrylene, ethyl hexyl methoxycinnamate, octyl salicylate, oxybenzone, padimate O, phenylbenzimidazole sulfonic acid, red petrolatum, sulisobenzone, titanium dioxide, trolamine salicylate, and mixtures thereof.

Some examples of UV absorbers are acetaminosalol, allatoin PABA, benzalphthalide, benzophenone, benzophenone 1-12, 3-benzylidene camphor, benzylidenecamphor hydrolyzed collagen sulfonamide, benzylidene camphor sulfonic Acid, benzyl salicylate, bornelone, bumetriozole, butyl Methoxydibenzoylmethane, butyl PABA, ceria/silica, ceria/silica talc, cinoxate, DEA-methoxycinnamate, dibenzoxazol naphthalene, di-t-butyl hydroxybenzylidene camphor, digalloyl trioleate, diisopropyl methyl cinnamate, dimethyl PABA ethyl cetearyldimonium tosylate, dioctyl butamido triazone, diphenyl carbomethoxy acetoxy naphthopyran, disodium bisethylphenyl tiamminotriazine stilbenedisulfonate, disodium distyrylbiphenyl triaminotriazine stilbenedisulfonate, disodium distyrylbiphenyl disulfonate, drometrizole, drometrizole trisiloxane, ethyl dihydroxypropyl PABA, ethyl diisopropylcinnamate, ethyl methoxycinnamate, ethyl PABA, ethyl urocanate, etrocrylene ferulic acid, glyceryl octanoate dimethoxycinnamate, glyceryl PABA, glycol salicylate, homosalate, isoamyl p-methoxycinnamate, isopropylbenzyl salicylate, isopropyl dibenzolylmethane, isopropyl methoxycinnamate, menthyl anthranilate, menthyl salicylate, 4-methylbenzylidene, camphor, octocrylene, octrizole, octyl dimethyl PABA, ethyl hexyl methoxycinnamate, octyl salicylate, octyl triazone, PABA, PEG-25 PABA, pentyl dimethyl

PABA, phenylbenzimidazole sulfonic acid, polyacrylamidomethyl benzylidene camphor, potassium methoxycinnamate, potassium phenylbenzimidazole sulfonate, red petrolatum, sodium phenylbenzimidazole sulfonate, sodium urocanate, TEA-phenylbenzimidazole sulfonate, TEA-salicylate, terephthalylidene dicamphor sulfonic acid, titanium dioxide, triPABA panthenol, urocanic acid, VA/crotonates/methacryloxybenzophenone-1 copolymer, and mixtures thereof.

Examples of antidandruff agents include pyridinethione salts, selenium compounds such as selenium disulfide, and soluble antidandruff agents, and mixtures thereof.

Examples of antiperspirant agents and deodorant agents include aluminum chloride, aluminum zirconium tetrachlorohydrex GLY, aluminum zirconium tetrachlorohydrex PEG, aluminum chlorohydrex, aluminum zirconium tetrachlorohydrex PG, aluminum chlorohydrex PEG, aluminum zirconium trichlorohydrate, aluminum chlorohydrex PG, aluminum zirconium trichlorohydrex GLY, hexachlorophene, benzalkonium chloride, aluminum sesquichlorohydrate, sodium bicarbonate, aluminum sesquichlorohydrex PEG, chlorophyllin-copper complex, triclosan, aluminum zirconium octachlorohydrate, zinc ricinoleate, and mixtures thereof.

Examples of skin protectants include allantoin, aluminium acetate, aluminium hydroxide, aluminium sulfate, calamine, cocoa butter, cod liver oil, colloidal oatmeal, dimethicone, glycerin, kaolin, lanolin, mineral oil, petrolatum, shark liver oil, sodium bicarbonate, talc, witch hazel, zinc acetate, zinc carbonate, zinc oxide, and mixtures thereof.

Examples of hair dyes include 1-acetoxy-2-methylnaphthalene; acid dyes; 5-amino-4-chloro-o-cresol; 5-amino-2,6-dimethoxy-3-hydroxypyridine; 3-amino-2,6-dimethylphenol; 2-amino-5-ethylphenol HCl; 5-amino-4-fluoro-2-methylphenol sulfate; 2-amino-4-hydroxyethylaminoanisole; 2-amino-4-hydroxyethylaminoanisole sulfate; 2-amino-5-nitrophenol; 4-amino-2-nitrophenol; 4-amino-3-nitrophenol; 2-amino-4-nitrophenol sulfate; m-aminophenol HCl; p-aminophenol HCl; m-aminophenol; o-aminophenol; 4,6-bis(2-hydroxyethoxy)-m-phenylenediamine HCl; 2,6-bis(2-hydroxyethoxy)-3,5-pyridinediamine HCl; 2-chloro-6-ethylamino-4-nitrophenol; 2-chloro-5-nitro-N-hydroxyethyl p-phenylenediamine; 2-chloro-p-phenylenediamine; 3,4-diaminobenzoic acid; 4,5-diamino-1-((4-chlorophenyl)methyl)-1H-pyrazole-sulfate; 2,3-diaminodihydropyrazolo pyrazolone dimethosulfonate; 2,6-diaminopyridine; 2,6-diamino-3-((pyridin-3-yl)azo)pyridine; dihydroxyindole; dihydroxyindoline; N,N-dimethyl-p-phenylenediamine; 2,6-dimethyl-p-phenylenediamine; N,N-dimethyl-p-phenylenediamine sulfate; direct dyes; 4-ethoxy-m-phenylenediamine sulfate; 3-ethylamino-p-cresol sulfate; N-ethyl-3-nitro PABA; gluconamidopropyl aminopropyl dimethicone; Haematoxylon brasiletto wood extract; HC dyes; Lawsonia inermis (Henna) extract; hydroxyethyl-3,4-methylenedioxyaniline HCl; hydroxyethyl-2-nitro-p-toluidine; hydroxyethyl-p-phenylenediamine sulfate; 2-hydroxyethyl picramic acid; hydroxypyridinone; hydroxysuccinimidyl C21-22 isoalkyl acidate; isatin; Isatis tinctoria leaf powder; 2-methoxymethyl-p-phenylenediamine sulfate; 2-methoxy-p-phenylenediamine sulfate ; 6-methoxy-2,3-pyridinediamine HCl; 4-methylbenzyl 4,5-diamino pyrazole sulfate; 2,2′-methylenebis 4-aminophenol; 2,2′-methylenebis-4-aminophenol HCl; 3,4-methylenedioxyaniline; 2-methylresorcinol; methylrosanilinium chloride; 1,5-naphthalenediol; 1,7-naphthalenediol; 3-nitro-p-Cresol; 2-nitro-5-glyceryl methylaniline; 4-nitroguaiacol; 3-nitro-p-hydroxyethylaminophenol; 2-nitro-N-hydroxyethyl-p-anisidine; nitrophenol; 4-nitrophenyl aminoethylurea; 4-nitro-o-phenylenediamine dihydrochloride; 2-nitro-p-phenylenediamine dihydrochloride; 4-nitro-o-phenylenediamine HCl; 4-nitro-m-phenylenediamine; 4-nitro-o-phenylenediamine; 2-nitro-p-phenylenediamine; 4-nitro-m-phenylenediamine sulfate; 4-nitro-o-phenylenediamine sulfate; 2-nitro-p-phenylenediamine sulfate; 6-nitro-2,5-pyridinediamine; 6-nitro-o-toluidine; PEG-3 2,2′-di-p-phenylenediamine; p-phenylenediamine HCl; p-phenylenediamine sulfate; phenyl methyl pyrazolone; N-phenyl-p-phenylenediamine HCl; pigment blue 15:1; pigment violet 23; pigment yellow 13; pyrocatechol; pyrogallol; resorcinol; sodium picramate; sodium sulfanilate; solvent yellow 85; solvent yellow 172; tetraaminopyrimidine sulfate; tetrabromophenol blue; 2,5,6-triamino-4-pyrimidinol sulfate; 1,2,4-trihydroxybenzene.

Example of nail care ingredients include butyl acetate; ethyl acetate; nitrocellulose; acetyl tributyl citrate; isopropyl alcohol; adipic acid/neopentyl glycol/trimelitic anhydride copolymer; stearalkonium bentonite; acrylates copolymer; calcium pantothenate; Cetraria islandica extract; Chondrus crispus; styrene/acrylates copolymer; trimethylpentanediyl dibenzoate-1; polyvinyl butyral; N-butyl alcohol; propylene glycol; butylene glycol; mica; silica; tin oxide; calcium borosilicate; synthetic fluorphlogopite; polyethylene terephtalate; sorbitan laurate derivatives; talc; jojoba extract; diamond powder; isobutylphenoxy epoxy resin; silk powder; and mixtures thereof.

Examples of fragrances or perfume include hexyl cinnamic aldehyde; anisaldehyde; methyl-2-n-hexyl-3-oxo-cyclopentane carboxylate; dodecalactone gamma; methylphenylcarbinyl acetate; 4-acetyl-6-tert-butyl-1,1-dimethyl indane; patchouli; olibanum resinoid; labdanum; vetivert; copaiba balsam; fir balsam; 4-(4-hydroxy-4-methyl pentyl)-3-cyclohexene-1-carboxaldehyde; methyl anthranilate; geraniol; geranyl acetate; linalool; citronellol; terpinyl acetate; benzyl salicylate; 2-methyl-3-(p-isopropylphenyl)-propanal; phenoxyethyl isobutyrate; cedryl acetal; aubepine; musk fragrances; macrocyclic ketones; macrolactone musk fragrances; ethylene brassylate; and mixtures thereof. Further perfume ingredients are described in detail in standard textbook references such as Perfume and Flavour Chemicals, 1969, S. Arctander, Montclair, N.J.

Examples of antioxidants are acetyl cysteine, arbutin, ascorbic acid, ascorbic acid polypeptide, ascorbyl dipalmitate, ascorbyl methylsilanol pectinate, ascorbyl palmitate, ascorbyl stearate, BHA, p-hydroxyanisole, BHT, t-butyl hydroquinone, caffeic acid, Camellia sinensis Oil, chitosan ascorbate, chitosan glycolate, chitosan salicylate, chlorogenic acids, cysteine, cysteine HCl, decyl mercaptomethylimidazole, erythorbic acid, diamylhydroquinone, di-t-butylhydroquinone, dicetyl thiodipropionate, dicyclopentadiene/t-butylcresol copolymer, digalloyl trioleate, dilauryl thiodipropionate, dimyristyl thiodipropionate, dioleyl tocopheryl methylsilanol, isoquercitrin, diosmine, disodium ascorbyl sulfate, disodium rutinyl disulfate, distearyl thiodipropionate, ditridecyl thiodipropionate, dodecyl gallate, ethyl ferulate, ferulic acid, hydroquinone, hydroxylamine HCl, hydroxylamine sulfate, isooctyl thioglycolate, kojic acid, madecassicoside, magnesium ascorbate, magnesium ascorbyl phosphate, melatonin, methoxy-PEG-7 rutinyl succinate, methylene di-t-butylcresol, methylsilanol ascorbate, nordihydroguaiaretic acid, octyl gallate, phenylthioglycolic acid, phloroglucinol, potassium ascorbyl tocopheryl phosphate, thiodiglycolamide, potassium sulfite, propyl gallate, rosmarinic acid, rutin, sodium ascorbate, sodium ascorbyl/cholesteryl phosphate, sodium bisulfite, sodium erythorbate, sodium metabisulfide, sodium sulfite, sodium thioglycolate, sorbityl furfural, tea tree (Melaleuca aftemifolia) oil, tocopheryl acetate, tetrahexyldecyl ascorbate, tetrahydrodiferuloylmethane, tocopheryl linoleate/oleate, thiodiglycol, tocopheryl succinate, thiodiglycolic acid, thioglycolic acid, thiolactic acid, thiosalicylic acid, thiotaurine, retinol, tocophereth-5, tocophereth-10, tocophereth-12, tocophereth-18, tocophereth-50, tocopherol, tocophersolan, tocopheryl linoleate, tocopheryl nicotinate, tocoquinone, o-tolyl biguanide, tris(nonylphenyl) phosphite, ubiquinone, zinc dibutyldithiocarbamate, and mixtures thereof.

Examples of oxidizing agents are ammonium persulfate, calcium peroxide, hydrogen peroxide, magnesium peroxide, melamine peroxide, potassium bromate, potassium caroate, potassium chlorate, potassium persulfate, sodium bromate, sodium carbonate peroxide, sodium chlorate, sodium iodate, sodium perborate, sodium persulfate, strontium dioxide, strontium peroxide, urea peroxide, zinc peroxide, and mixtures thereof.

Examples of reducing agents are ammonium bisufite, ammonium sulfite, ammonium thioglycolate, ammonium thiolactate, cystemaine HCl, cystein, cysteine HCl, ethanolamine thioglycolate, glutathione, glyceryl thioglycolate, glyceryl thioproprionate, hydroquinone, p-hydroxyanisole, isooctyl thioglycolate, magnesium thioglycolate, mercaptopropionic acid, potassium metabisulfite, potassium sulfite, potassium thioglycolate, sodium bisulfite, sodium hydrosulfite, sodium hydroxymethane sulfonate, sodium metabisulfite, sodium sulfite, sodium thioglycolate, strontium thioglycolate, superoxide dismutase, thioglycerin, thioglycolic acid, thiolactic acid, thiosalicylic acid, zinc formaldehyde sulfoxylate, and mixtures thereof.

Examples of propellant gases include carbon dioxide, nitrogen, nitrous oxide, volatile hydrocarbons such as butane, isobutane, or propane, and chlorinated or fluorinated hydrocarbons such as dichlorodifluoromethane and dichlorotetrafluoroethane or dimethylether; and mixtures thereof.

Examples of film formers include those polymers capable, by themselves or in the presence of an auxiliary film-forming agent, of forming a macroscopically continuous film on a support, especially on keratin materials, preferably a cohesive film and better still a film whose cohesion and mechanical properties are such that the said film can be isolated from the said support.

Examples of antiacne agents include salicylic acid, sulfur benzoyl, peroxide, tretinoin, and mixtures thereof.

Examples of antibacterial agents include chlorohexadiene gluconate, alcohol, benzalkonium chloride, benzethonium chloride, hydrogen peroxide, methylbenzethonium chloride, phenol, poloxamer 188, povidone-iodine, and mixtures thereof.

Examples of antifungal agents include miconazole nitrate, calcium undecylenate, undecylenic acid, zinc undecylenate, and mixtures thereof.

Examples of therapeutic active agents include penicillins, cephalosporins, tetracyclines, macrolides, epinephrine, amphetamines, aspirin, acetominophen, barbiturates, catecholamines, benzodiazepine, thiopental, codeine, morphine, procaine, lidocaine, benzocaine, sulphonamides, ticonazole, perbuterol, furosamide, prazosin, hormones, prostaglandins, carbenicillin, salbutamol, haloperidol, suramin, indomethicane, diclofenac, glafenine, dipyridamole, theophylline, hydrocortisone, steroids, scopolamine, and mixtures thereof.

Examples of external analgesics are benzyl alcohol, capsicum oleoresin (Capsicum frutescens oleoresin), methyl salicylate, camphor, phenol, capsaicin, juniper tar (Juniperus oxycedrus tar), phenolate sodium (sodium phenoxide), capsicum (Capsicum frutescens), menthol, resorcinol, methyl nicotinate, turpentine oil (turpentine), and mixtures thereof.

An example of a skin bleaching agent is hydroquinone.

Examples of anti-cancer agents include alkylating agents (such as busulfan, fluorodopan), antimitotic agents (such as colchicine, rhizoxin), topoisomerase I inhibitors (such as camptothecin and its derivatives), topoisomerase II inhibitors (such as menogaril, amonafide), RNA/DNA or DNA anti-metabolites (such as acivicin, guuanazole), plant alkaloids and terpenoids, antineoplastics, some plant-derived compounds (such as podophyllotoxin, vinca alkaloids), and mixtures thereof.

Examples of diuretics include loop diuretics (such as bumetanide, furosemide), thiazide diuretics (such as chlorothiazide, hydroflumethiazide), potassium-sparing diuretics (such as amioloride, spironolactone), carbonic anhydrase inhibitors (such as acetazolamide), osmotic diuretics (such as mannitol), and mixtures thereof.

Examples of agents for treating gastric and duodenal ulcers include proton pump inhibitor (such as lansoprazole, omeprazole), acid blockers or H2 histamine blockers (such as cimetidine, ranitidine), bismuth, sucralfate, and mixtures thereof.

Examples of proteolytic enzymes include nattokinase, serratiopeptidase, bromelain, papain, and mixtures thereof.
Examples of antihistamine or H1 histamine blockers include brompheniramine, clemastine, cetirizine, loratadine, fexofenadine, and mixtures thereof.

Examples of sedatives include barbiturates (such as phenobarbitol), benzodiazepines (such as lorazepam), herbal sedatives, benzodiazepine-like drugs (such as zolpidem, zopiclone), and mixtures thereof.

Examples of bronchodilators include short-acting β2-agonists and long-acting β2-agonists, anticholinergics, and mixtures thereof.

The formulations of the present invention also include diluents. Such diluents are often necessary to decrease the viscosity of the formulation sufficiently for application.

Examples of diluents include silicon containing diluents such as hexamethyldisiloxane, octamethyltrisiloxane, and other short chain linear siloxanes such as octamethyltrisiloxane, decamethyltetrasiloxane, dodecamethylpentasiloxane, tetradecamethylhexasiloxane, hexadeamethylheptasiloxane, heptamethyl-3-{(trimethylsilyl)oxy)}trisiloxane, cyclic siloxanes such as hexamethylcyclotrisiloxane, octamethylcyclotetrasiloxane, decamethylcyclopentasiloxane, dodecamethylcyclohexasiloxane; organic diluents such as butyl acetate, alkanes, alcohols, ketones, esters, ethers, glycols, glycol ethers, hydrofluorocarbons or any other material which can dilute the formulation without adversely affecting any of the component materials of the cosmetic composition. Hydrocarbons include isododecane, isohexadecane, Isopar L (C11-C13), Isopar H (C11-C12), hydrogenated polydecene. Ethers and esters include isodecyl neopentanoate, neopentylglycol heptanoate, glycol distearate, dicaprylyl carbonate, diethylhexyl carbonate, propylene glycol n butyl ether, ethyl-3 ethoxypropionate, propylene glycol methyl ether acetate, tridecyl neopentanoate, propylene glycol methylether acetate (PGMEA), propylene glycol methylether (PGME), octyldodecyl neopentanoate, diisobutyl adipate, diisopropyl adipate, propylene glycol dicaprylate/dicaprate, and octyl palmitate. Additional organic diluents include fats, oils, fatty acids, and fatty alcohols.

Further materials suitable for the personal care and health care are well known to the person skilled in the art and are described in many text books as well as other publications.

The general level of bi-modal water continuous emulsion (E) in the cosmetic compositions may vary from 0.1% to 80% by weight, alternatively from 0.2% to 10%, alternatively from 0.5% to 5%, relative to the total weight of the cosmetic composition. The cosmetic ingredient (C) is present at a level of from 0.01% to 99.99% by weight, relative to the total weight of the cosmetic composition. The cosmetic ingredient (C) may be a mixture of cosmetic ingredients (C) as listed above.

In some instances, the bi-modal water continuous emulsion (E) is used in conjunction with a cosmetic ingredient (C) selected from a cationic conditioning agent, a hydrophobic conditioning agent, or mixtures thereof, in a cosmetically acceptable medium.

The cosmetic composition may be prepared by a process comprising the steps of
i. Mixing a bi-modal water continuous emulsion (E) comprising at least 70 weight percent of: a first dispersed phase (i) containing a hydrophobic oil, wherein the hydrophobic oil is provided as a non-emulsified hydrophobic oil, a second dispersed phase (ii) containing a silicone, wherein the silicone is provided from a water continuous silicone emulsion containing at least one surfactant; in an aqueous phase
ii. and at least one cosmetic ingredient (C),
iii. optionally in the presence of a cosmetically acceptable medium.

The cosmetic compositions may be prepared by mixing the bi-modal water continuous emulsion (E) in the aqueous phase with the appropriate aqueous phase ingredients, and optionally provide for a non aqueous phase, and mix the aqueous and non aqueous phases together, optionally under heating.

The process may be conducted at temperatures ranging of from 15 to 90° C., alternatively of from 20 to 60° C., alternatively at room temperature (25° C.), using simple propeller mixers, counter-rotating mixers, or homogenizing mixers. No special equipment or processing conditions are typically required. Depending on the type of composition prepared, the method of preparation will be different, but such methods are well known in the art.

The cosmetic compositions may be in the form of a cream, a gel, a powder (free flowing powder or pressed), a paste, a solid, freely pourable liquid, an aerosol. The cosmetic compositions may be in the form of monophasic systems, biphasic or alternate multiphasic systems; emulsions, e.g. oil-in-water, water-in-oil, silicone-in-water, water-in-silicone; multiple emulsions, e.g. oil-in-water-in-oil, polyol-in-silicone-in-water, oil-in-water-in-silicone.

Skin care compositions include shower gels, soaps, hydrogels, creams, lotions and balms; antiperspirants; deodorants such as sticks, soft solid, roll on, aerosol, and pump sprays; skin creams; skin care lotions; moisturizers; facial treatments such as wrinkle control or diminishment treatments; exfoliates; body and facial cleansers; bath oils; perfumes; colognes; sachets; sunscreens; mousses; patches; pre-shave and after-shave lotions; shaving soaps; shaving lathers; depilatories; make-ups; color cosmetics; foundations; concealers; blushes; lipsticks; eyeliners; mascaras; oil removers; color cosmetic removers, powders, and kits thereof.

Hair care compositions include shampoos, rinse-off conditioners, leave-in conditioners and styling aids, gels, sprays, pomades, mousses, waxes, cuticle coats, hair colorants, hair relaxants, hair straighteners, permanents, and kits thereof.

Nail care compositions include color coats, base coats, nail hardeners, and kits thereof.

Health care compositions may be in the form of ointments, creams, gels, mousses, pastes, patches, spray on bandages, foams and/or aerosols or the like, medicament creams, pastes or sprays including anti-acne, dental hygienic, antibiotic, healing promotive, which may be preventative and/or therapeutic medicaments, and kits thereof.

The cosmetic compositions may be used by the standard methods, such as applying them to the human or animal body, e.g. skin or hair, using applicators, brushes, applying by hand, pouring them and/or possibly rubbing or massaging the composition onto or into the body. Removal methods, for example for colour cosmetics are also well known standard methods, including washing, wiping, peeling and the like.

The invention also comprises a method of treating keratinous substrates, such as hair or skin, by applying to it a cosmetic composition according to the first aspect of the invention.

The cosmetic compositions may be used on hair in a conventional manner. An effective amount of the composition for washing or conditioning hair is applied to the hair. Such effective amounts generally range from about lg to about 50 g, preferably from about lg to about 20 g. Application to the hair typically includes working the cosmetic composition through the hair such that most or all of the hair is contacted with the cosmetic composition. These steps can be repeated as many times as desired to achieve the desired benefit.

Benefits obtained from using the cosmetic compositions on hair include one or more of the following benefits: hair conditioning, softness, detangling ease, silicone deposition, anti-static, anti-frizz, lubricity, shine, strengthening, viscosity, tactile, wet combing, dry combing, improvement in coloration process, color retention, straightening, heat protection, styling, or curl retention.

The cosmetic compositions may be used on skin in a conventional manner. An effective amount of the composition for the purpose is applied to the skin. Such effective amounts generally range from about 1 mg/cm² to about 3 mg/cm². Application to the skin typically includes working the cosmetic composition into the skin. This method for applying to the skin comprises the steps of contacting the skin with the cosmetic composition in an effective amount and then rubbing the composition into the skin. These steps can be repeated as many times as desired to achieve the desired benefit.

Benefits obtained from using the cosmetic compositions on skin include one or more of the following benefits: skin softness, suppleness, moisturisation, skin feel, foam generation.
A process of washing keratinous fibres comprises the steps of
i. Mixing a bi-modal water continuous emulsion (E) comprising at least 70 weight percent of: a first dispersed phase containing a hydrophobic oil, wherein the hydrophobic oil is provided as a non-emulsified hydrophobic oil, a second dispersed phase containing a silicone, wherein the silicone is provided from a water continuous silicone emulsion containing at least one surfactant;
   with at least one cosmetic ingredient (C) optionally in the presence of a cosmetically acceptable medium,
ii. Applying the mixture to the keratinous fibres;
iii. Optionally let the mixture stand on the keratinous fibres;
iv. Optionally rinsing the keratinous fibres.

A process of conditioning keratinous fibres comprises the steps of
i. Mixing a bi-modal water continuous emulsion (E) comprising at least 70 weight percent of: a first dispersed phase containing a hydrophobic oil, wherein the hydrophobic oil is provided as a non-emulsified hydrophobic oil, a second dispersed phase containing a silicone, wherein the silicone is provided from a water continuous silicone emulsion containing at least one surfactant;
   with at least one cosmetic ingredient (C) selected from cationic conditioning polymer, hydrophobic conditioning polymer, optionally in the presence of a cosmetically acceptable medium,
ii. Applying the mixture to the keratinous fibres;
iii. Optionally let the mixture stand on the keratinous fibres;
iv. Optionally rinsing the keratinous fibres.

The optional standing time of the process of caring or conditioning keratinous fibres may range of from 10 seconds to 1 hour, alternatively of from 30 seconds to 30 minutes, alternatively of from 30 seconds to 10 minutes.

The cosmetic composition may be used to care for keratinous substrates, that is to cleanse, to condition, to refresh, to make up, to remove make up, to fix hair.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention. All percentages are in wt. %. All measurements were conducted at 23° C. unless indicated otherwise.

INSTRON procedures are standard, recognized, and industrially acceptable protocols, see for example, U.S. Pat. No. 5,389,364 (Feb. 14, 1995), U.S. Pat. No. 5,409,695 (Apr. 25, 1995), U.S. Pat. No. 5,419,627 (May 30, 1995), and U.S. Pat. No. 5,504,149 (Apr. 2, 1996).

INSTRON COMBING is an industry recognized test for determining hair conditioning obtained from hair compositions, by the ease of wet combing and the ease of dry combing. The test employs an INSTRON strain gauge, which is equipped to measure the force required to comb the hair. The conditioning performance is based on the ability of a particular hair treatment formulation, such as a shampoo or a hair conditioner, to reduce the force required to comb the hair with the INSTRON strain gauge. The force is reported as an Average Combing Load (ACL). The lower the number of the ACL value, the better is the conditioning effect imparted by the formulation being tested. Typically, ACL baselines are initially established using untreated tresses that have only been washed with a sodium lauryl sulfate solution. The effectiveness of a treatment can then be expressed as an ACL of the treated tress or percent reduction in ACL, calculated using the relationship: (untreated hair ACL—treated hair ACL)×100 divided by the untreated hair ACL.

Slightly bleached European human hair from DeMeo Brothers was used for testing the compositions prepared herein. Each tress weighed about 2.0 grams. Each tress was rinsed for 15 seconds under a stream of 40° C. tap water. Using a pipette, 1.0 gram of a solution containing nine percent of sodium lauryl sulfate was applied and lathered through the tress for 30 seconds. The tress was rinsed for 30 seconds under running water. Excess water was removed from the tress by passing the tress between the index and middle fingers of the hand. The tresses were placed on a tray covered with paper towels and dried overnight. Each tress was hand combed three times with the narrow teeth of an ACE® comb, and evaluated using INSTRON WET and INSTRON DRY COMBING procedures.

For tests involving shampoo compositions, hair tresses are rinsed with tap water for 30 seconds at 40° C. The test composition is applied to the tress in the amount of 0.8 gram, and the tress is stroked for 30 seconds. The tress is rinsed for 30 seconds under tap water at 40° C. Excess water is removed by pulling the tress through the index and middle fingers of the hand. The tresses are allowed to dry separately on a paper towel overnight at room temperature. The tresses are combed once before performing an INSTRON study.

According to the INSTRON WET COMBING method, hair is first wetted by dipping it into distilled water, and then the hair is detangled by combing the tress three times. The tress is then retangled by dipping in distilled water three times. Excess water is removed by passing the tress through the index and middle fingers of the hand twice. The tress is placed on a hanger and INSTRON combed. Retangling and INSTRON combing are repeated until all data points are collected. An average combing force of three tresses is measured for each treatment.

According to the INSTRON DRY COMBING method, hair is detangled by combing the tress 3 times. Then hair is retangled by swirling the tress clockwise 3 times and swirling it counter clockwise 3 times. The tress is then placed on a hanger and INSTRON combed. Retangle and Instron combing are repeated until all data points are collected. An average combing force for three tresses is measured for each treatment.

### Emulsion 1

Emulsification of dimethicone 100,000 mPa·s (A) with an emulsion of dimethiconol and TEA-dodecylbenzenesulfonate (B—60% silicone): 32.74 g of 100,000 mPa·s dimethicone was weighed into a cup followed by 6.55 g of emulsion (B). The cup was closed and placed inside a DAC-150 SpeedMixer® and the cup was spun at maximum speed (3450 RPM) for 30 seconds. The cup was opened and the walls of the cup were scraped with a spatula and the cup was spun again at maximum speed for 30 seconds. 5 g of emulsion (B) was weighed into the cup and the cup was spun for 30 seconds at approximately 2500 RPM. 15.7 g of emulsion (B) was added and the cup was again spun for 30 seconds at approximately 2500 RPM. The resulting emulsion (E1) consisted of an aqueous oil-in-water (o/w) emulsion of silicone polymer having a total silicone content of approximately 81.05 percent. On a dry basis, this emulsion (E1) contained approximately 77 percent 100,000 mPa·s dimethicone as large particles and 23 percent dimethiconol as smaller particles. Particle size of the emulsion (E1) was determined using a Malvern Mastersizer®. The particle size curve showed two distinct peaks, one centred at 6.5 μm and another that was centred at 35 μm. Particle size as calculated by the instrument was as follows: Dv50=8.68 μm, Dv90=19.82 μm, Mode 1=0.73 μm, Mode 2=11.81 μm.

### Emulsion 2

Emulsification of dimethicone 100,000 mPa·s (A) with an emulsion of dimethiconol and TEA-dodecylbenzenesulfonate (B—60% silicone) and with siloxylated polyether EO/PO co-polymer: this example is processed as Example 1, with the addition of 2.99 g of siloxylated polyether EO/PO co-polymer. The particle size curve showed two distinct peaks, one centred at 6.5 μm and another that was centred at 35 μm. Particle size as calculated by the instrument was as follows: Dv50=9.3 μm, Dv90=21.16 μm, Mode 1=0.73 μm, Mode 2=12.38 μm.

### Comparative Emulsion 1

Emulsion of dimethiconol and TEA-dodecylbenzenesulfonate (B), 60% wt silicone

### Comparative Emulsion 2

Mixture of emulsions: emulsion of dimethiconol and TEA-dodecylbenzenesulfonate+emulsion dimethicone and cocamidopropyl betaine and C12-15 pareth-3 and guar hydroxypropyltrimonium chloride

Emulsions 1 and 2 and Comparative emulsions 1 and 2 were formulated in a shampoo composition at 2% wt silicone and compared for wet and dry combing performances.

Performances of Emulsions 1 and 2 in dry combing and wet combing (Instron) showed improvement over Comparative emulsion 1.

Performances of Emulsions 1 and 2 in dry combing and wet combing (Instron) showed equivalence with Comparative emulsion 2.

Sensorial benefits of shampoos containing Emulsions 1 and 2 when applied on hair were shown very good.

### Emulsions 3 to 6

These were prepared in a similar manner as Emulsions 1 and 2 above, with contents as listed in Table 1, and were used in shampoo compositions as listed in Table 2, containing each 2 wt % silicone. The shampoo compositions were subsequently evaluated per the Instron technique described above.

TABLE 1

| Material (%) | Emulsion 3 | Emulsion 4 | Emulsion 5 | Emulsion 6 |
|---|---|---|---|---|
| Dimethicone 600,000 mPa · s | 83.00 | 92.00 | 80.00 | 90.00 |
| Divinyldimethicone/Dimethicone | 15.00 | | | |
| Copolymer and C12-13 Pareth-23 | | | | |
| and C12-13 Pareth-3 (60% silicone | | | | |
| emulsion) (Comparative emulsion 3) | | | | |
| Dimethiconol (and) | | 6.00 | | |
| TEADodecylbenzenesulfonate (and) | | | | |
| Laureth-23 (25% silicone emulsion) | | | | |
| (Comparative emulsion 4) | | | | |
| Dimethiconol (and) TEA- | | | 20.00 | |
| Dodecylbenzenesulfonate (60% | | | | |
| silicone emulsion) (Comparative | | | | |
| emulsion 1) | | | | |
| Dimethiconol (and) TEA- | | | | 9.00 |
| Dodecylbenzenesulfonate (51% | | | | |
| silicone emulsion) (Comparative | | | | |
| emulsion 5) | | | | |
| Water | 2.00 | 2.00 | 0.00 | 1.00 |
| Total silicone (%) | 92.00 | 93.50 | 92.00 | 94.59 |
| Particle sizes | | | | |
| Primary Particle Size (microns) | 8.47 | 20.57 | 31.34 | 36.49 |
| Secondary Particle size (microns) | 0.953 | 0.697 | NM | NM |
| D(50) Particle Size (microns) | 8.73 | 20.76 | 28.08 | 36.71 |
| D(90) Particle Size (microns) | 17.89 | 44.92 | 67.25 | 89.86 |

| | [symbol] ABLE 2 | | | | | | | |
|---|---|---|---|---|---|---|---|---|
| | [symbol] ample | | | | | | | |
| | Example | Example | Example | Example | Comparative | Comparative | Comparative | Comparative |
| | Shampoo 1 | Shampoo 2 | Shampoo 3 | Shampoo 4 | Shampoo 1 | Shampoo 2 | Shampoo 3 | Shampoo 4 |
| [symbol] gredients | wt % | wt % | wt % | wt % | wt % | wt % | wt % | wt % |
| [symbol] hase A | | | | | | | | |
| [symbol] olyquaternium-10¹ | 0.3% | 0.3% | 0.3% | 0.3% | 0.3% | 0.3% | 0.3% | 0.3% |
| [symbol] odium Lauryl Ether | 30.0% | 30.0% | 30.0% | 30.0% | 30.0% | 30.0% | 30.0% | 30.0% |
| Sulfate² | | | | | | | | |
| PEG-150 Pentaerythrityl | 1.0% | 1.0% | 1.0% | 1.0% | 1.0% | 1.0% | 1.0% | 1.0% |
| Tetrastearate³ | | | | | | | | |
| Deionized Water | 56.1% | 56.2% | 56.1% | 56.2% | 55.0% | 50.3% | 55.0% | 54.4% |
| Phase B | | | | | | | | |
| Cocamide DEA⁴ | 3.0% | 3.0% | 3.0% | 3.0% | 3.0% | 3.0% | 3.0% | 3.0% |
| Cocamidopropyl Betaine⁵ | 7.0% | 7.0% | 7.0% | 7.0% | 7.0% | 7.0% | 7.0% | 7.0% |

Phase C: Silicone Emulsion

| | | | | | | | | |
|---|---|---|---|---|---|---|---|---|
| Emulsion 3 | 2.2% | | | | | | | |
| Emulsion 4 | | 2.1% | | | | | | |
| Emulsion 5 | | | 2.2% | | | | | |
| Emulsion 6 | | | | 2.1% | | | | |
| Divinyldimethicone/Dimeth- | | | | | 3.3% | | | |
| icone Copolymer and | | | | | | | | |
| C12-13 Pareth-23 and C12- | | | | | | | | |
| 13 Pareth-3 emulsion | | | | | | | | |
| (Comparative emulsion 3) | | | | | | | | |
| Dimethiconol and | | | | | | 8.0% | | |
| TEADodecylbenzenesulfo- | | | | | | | | |
| [symbol] ate and Laureth-23 | | | | | | | | |
| [symbol] mulsion (Comparative | | | | | | | | |
| [symbol] mulsion 4) | | | | | | | | |
| [symbol] imethiconol and TEA- | | | | | | | 3.3% | |
| [symbol] odecylbenzenesulfonate | | | | | | | | |
| [symbol] 0% silicone emulsion) | | | | | | | | |
| [symbol] omparative emulsion 1) | | | | | | | | |
| [symbol] imethiconol and TEA- | | | | | | | | 3.9% |
| [symbol] odecylbenzenesulfonate | | | | | | | | |
| (51% silicone emulsion) | | | | | | | | |
| (Comparative emulsion 5) | | | | | | | | |
| Phase D | | | | | | | | |
| Deionized Water | q.s. | q.s. | q.s. | q.s. | q.s. | q.s. | q.s. | q.s. |
| DMDM Hydantoin⁶ | 0.4% | 0.4% | 0.4% | 0.4% | 0.4% | 0.4% | 0.4% | 0.4% |

Performance - INSTRON

| | | | | | | | | |
|---|---|---|---|---|---|---|---|---|
| Wet combing reduction | 35.3 | 28.8 | 49.1 | 65.4 | 48.6 | 74.5 | 46.1 | 69.4 |
| (%) | | | | | | | | |
| Dry combing reduction (%) | 49.5 | 45.3 | 43.6 | 29.2 | 58.6 | 54.8 | 40.5 | 54.6 |

H&H Ref.: 071049.00388
DCC Ref.: DC11633 PCT 1
1. UCARE Polymer JR-30M available from Dow Chemical
2. Standapol ES-3 ® available from Cognis Corp.
3. Crothix ® available from Croda Inc.
4. Monamid 705 ® available from Croda Inc.
5. Monateric CAB-LC ® available from Croda Inc.
6. Glydant ® available from Lonza, Inc. [symbol]
indicates data missing or illegible when filed

Shampoos preparation (Table 2): deionized water was added to the mixing vessel. In order to keep the active silicone loading constant throughout testing, it was necessary to adjust the water level added depending on the percent active silicone in the various emulsions used. With moderate agitation, the polyquaternium-10 was dispersed until fully dissolved and sodium lauryl ether sulfate added. This was then heated to 75° C. and the PEG-150 pentaerythrityl tetrastearate was added with continual mixing. Heat was decreased to 40° C. and cocamide DEA and cocamidopropyl betaine were added. When completely incorporated, silicone emulsion was added to the base shampoo. The shampoo was mixed for 5-10 minutes and then DMDM hydantoin was added. The water loss was compensated for (to 100% wt total) and the formulation was mixed for an additional 5 minutes. The final pH of the shampoo formulations were ranging of from 5.5 to 6.0.

The results of INSTRON WET COMBING and DRY COMBING using shampoos from Table 2 were also shown in Table 2.

INSTRON WET COMBING: The results show that all Example shampoos 1 to 4 containing the bi-modal Emulsions 3 to 6 respectively, provided a reduction in wet combing force. The Example shampoos 1, 3 and 4 were similar in performance to the respective Comparative shampoos 1, 3 and 4, meaning they are capable of improving the wet conditioning properties of hair.

INSTRON DRY COMBING: The results show that all Example shampoos 1 to 4 containing the bi-modal Emulsions 3 to 6 respectively, provided a reduction in dry combing force. The Example shampoos 1 to 4 were similar in performance to the respective Comparative shampoos 1 to 4, meaning they are capable of improving the dry conditioning properties of hair.

Wet feel/slipperiness, dry feel/slipperiness, smoothness, shine and volume of the tresses treated with the Example shampoos 1 to 4 were similar to the tresses treated with the respective Comparative shampoos 1 to 4.

### Emulsion 7

Emulsification of (A) as a mixture of avocado oil, hydrogenated polyisobutene, dimethicone and dimethicone crosspolymer, with an emulsion (B) of dimethicone 60,000 mPa·s and laureth-4 and laureth-23 (B—58.5% silicone): 8.15 g of emulsion (B), 3.15 g of avocado oil and 3.15 g of hydrogenated polyisobutene, were weighed into a cup. The cup was closed and placed inside a DAC-150 SpeedMixer® and the cup was spun at maximum speed (3450 RPM) for 60 seconds. 6.30 g of dimethicone and dimethicone crosspolymer were added to the cup and the cup was spun for 60 seconds at 3450 RPM. 1.66 g of glass beads were added to provide additional shear, and the mixture was spun for 60 seconds at 3450 RPM. 0.50 g water was added and the mixture was spun for 30 seconds at 3450 RPM. An additional 0.50 g water was added and the mixture was spun for 30 seconds at 3450 RPM. A further additional 0.74 g water was added and the mixture was spun for 30 seconds at 3450 RPM. The bulk viscosity of the emulsion was reduced from unflowable to flowable after addition of the additional 0.5 g and 0.74 g water.

The resulting emulsion (E7) consisted of an aqueous oil-in-water (o/w) emulsion of silicone polymer and organic oil having a total internal phase content (silicone+organic oil) of approximately 81.78% wt. On a dry basis, this emulsion (E7) contained approximately 27.5% 60,000 mPa·s dimethicone, 18.1% avocado oil, 18.1% hydrogenated polyisobutene, and 36.3% dimethicone and dimethicone crosspolymer. Particle size of the emulsion (E7) was determined using a Malvern Mastersizer®. Particle size as calculated by the instrument was as follows: Dv50=2.095μm, Dv90=21.162μm, Mode 1=1.458 μm, Mode 2=11.294 μm.

### Emulsion 8

Emulsification of (A) as a mixture of laureth-7, pentaerythrityl tetraethylhexanoate, hydrogenated polyisobutene, with an emulsion (B) of dimethicone and dimethicone/vinyl dimethicone crosspolymer and PEG-20 sorbitan cocoate (B—65% silicone): 8.00 g of emulsion (B), 0.16 g laureth-7, 4.00 g pentaerythrityl tetraethylhexanoate, 4.00 g hydrogenated polyisobutene, were weighed into a cup. The cup was closed and placed inside a DAC-150 SpeedMixer® and the cup was spun at maximum speed (3450 RPM) for 30 seconds. 0.50 g water was added and the mixture was spun for 30 seconds at 3450 RPM. The resulting emulsion (E8) consisted of an aqueous oil-in-water (o/w) emulsion of silicone polymer and organic oil having a total internal phase content (silicone+organic oil) of approximately 78.73% wt. On a dry basis, this emulsion (E8) contained approximately 39.0% dimethicone and dimethicone/Vinyl dimethicone crosspolymer, 30.5% pentaerythrityl tetraethylhexanoate, and 30.5% hydrogenated polyisobutene. Particle size of the emulsion (E8) was determined using a Malvern Mastersizer®. Particle size as calculated by the instrument was as follows: Dv50=3.488 μm, Dv90=6.903 μm, Mode 1=1.02 μm, Mode 2=4.689 μm.

Although optional, the laureth-7 may be replaced by laureth-4, laureth-23, polysorbate-20 or trideceth-9.

The emulsion (B) of dimethicone and dimethicone/vinyl dimethicone crosspolymer and PEG-20 sorbitan cocoate may be replaced by an emulsion (B) of dimethicone and dimethicone/vinyl dimethicone crosspolymer and ceteth-6 and ceteth-20.

Additional water might be added to reduce the bulk viscosity of the emulsion.

### Emulsion 9

Emulsification of (A) as a mixture of bis-diisopropanolamino-PG-propyl dimethicone/bis-isobutyl PEG-14 copolymer and polysorbate 20, with an emulsion (B) of bis-diisopropanolamino-PG-propyl dimethicone/bis-isobutyl PEG-14 copolymer and polysorbate 20 and butyloctanol (B—56% silicone): 10.00 g of emulsion (B), 10.00 g of bis-diisopropanolamino-PG-propyl dimethicone/bis-isobutyl PEG-14 copolymer and 0.05 g of polysorbate 20, were weighed into a cup. The cup was closed and placed inside a DAC-150 SpeedMixer® and the cup was spun at maximum speed (3450 RPM) for 30 seconds. An additional 5.00 g of emulsion (B) was added and the mixture was spun for 30 seconds at 3450 RPM.

The resulting emulsion (E9) consisted of an aqueous oil-in-water (o/w) emulsion of silicone polymer having a total silicone content of approximately 74.31% wt. The entire internal phase (small and large particles) is bis-diisopropanolamino-PG-propyl dimethicone/bis-isobutyl PEG-14 copolymer. Particle size of the emulsion (E9) was determined using a Malvern Mastersizer®. Particle size as calculated by the instrument was as follows: Dv50=6.059 μm, Dv90=11.395 μm, Mode 1=0.924 μm, Mode 2=6.250 μm.

### Emulsion 10

Emulsification of (A) as a mixture of dimethiconol 80 Pa·s, laureth-4 and laureth-23, with an emulsion (B) of bis-diisopropanolamino-PG-propyl dimethicone/bis-isobutyl PEG-14 copolymer and polysorbate 20 and butyloctanol (B—56% silicone): 8.00 g of emulsion (B), 42.86 g of dimethiconol, 0.03 g of laureth-4 and 0.08 g of laureth-23 (70% active solution), were weighed into a cup. The cup was closed and placed inside a DAC-150 SpeedMixer® and the cup was spun at maximum speed (3450 RPM) for 30 seconds. A first additional 4.00 g of emulsion (B) was added and the mixture was spun for 30 seconds at 3450 RPM. A second additional 4.00 g of emulsion (B) was added and the mixture was spun for 30 seconds at 3450 RPM. A third additional 4.00 g of emulsion (B) was added and the mixture was spun for 30 seconds at 3450 RPM. A fourth additional 4.00 g of emulsion (B) was added and the mixture was spun for 30 seconds at 3450 RPM. A last additional 10.00 g of emulsion (B) was added and the mixture was spun for 30 seconds at 3450 RPM.

The resulting emulsion (E10) consisted of an aqueous oil-in-water (o/w) emulsion of silicone polymer having a total silicone content of approximately 81.05% wt. On a dry basis, this emulsion (E10) contained approximately 68.6 percent 80 Pa·s dimethiconol as large particles and 31.4% wt bis-diisopropanolamino-PG-propyl dimethicone/bis-isobutyl PEG-14 copolymer as smaller particles. Particle size of the emulsion (E10) was determined using a Malvern Mastersizer®. Particle size as calculated by the instrument was as follows: Dv50=7.041μm, Dv90=14.265μm, Mode 1=1.045 μm, Mode 2=7.665 μm.

### Emulsion 11

Emulsification of (A) as dimethiconol 80 Pa·s, with an (micro)emulsion (B) of silicone quaternium-16 and undeceth-11 and butyloctanol and undeceth-5 (B—22% silicone—Dv50=0.0463 μm): 5.00 g of emulsion (B) and 28.20 g of dimethiconol were weighed into a cup. 10.00 g of glass beads were added to provide additional shear. The cup was closed and placed inside a DAC-150 SpeedMixer® and the cup was spun at maximum speed (3450 RPM) for 30 seconds. A first additional 5.00 g of emulsion (B) was added and the mixture was spun for 30 seconds at 3450 RPM. A second additional 2.50 g of emulsion (B) was added and the mixture was spun for 30 seconds at 3450 RPM. A third additional 2.50 g of emulsion (B) was added and the mixture was spun for 30 seconds at 3450 RPM.

The resulting emulsion (E11) consisted of an aqueous oil-in-water (o/w) emulsion of silicone polymer having a total silicone content of approximately 75.00% wt. On a dry basis, this emulsion (E11) contained approximately 87.0% wt dimethiconol as large particles and 13.0% wt silicone quaternium-16 as smaller particles. Particle size of the emulsion (E11) was determined using a Malvern Mastersizer®, for only the larger particles with Mode 2=6.579 μm. The Dv50 of the initial emulsion (B), as measured by a Microtrac Nanotrac 150, is of 0.0463 μm. These smaller particles cannot effectively be measured when the larger particles are present, because of the optical effect of the larger particles on the measurement apparatus. Nevertheless, evidence of the presence of such smaller particles is made by the flowable character of Emulsion 11 which would not be the case if only large particles were present at such high silicone content.

### Emulsion 12

Emulsification of (A) as dimethiconol 80 Pa·s, with a (micro)emulsion (B) of amodimethicone and) C11-15 pareth-7 and) laureth-9 and glycerin and trideceth-12 (B—20% silicone—Dv50=0.0146 μm): 2.50 g of emulsion (B) and 27.74 g of dimethiconol were weighed into a cup. 10.00 g of glass beads were added to provide additional shear. The cup was closed and placed inside a DAC-150 SpeedMixer® and the cup was spun at maximum speed (3450 RPM) for 30 seconds. A first additional 2.50 g of emulsion (B) was added and the mixture was spun for 30 seconds at 3450 RPM. A further additional 5.00 g of emulsion (B) was added and the mixture was spun for 30 seconds at 3450 RPM. A last additional 2.50 g of emulsion (B) was added and the mixture was spun for 30 seconds at 3450 RPM.

The resulting emulsion (E12) consisted of an aqueous oil-in-water (o/w) emulsion of silicone polymer having a total silicone content of approximately 75.15% wt. On a dry basis, this emulsion (E12) contained approximately 91.7% wt dimethiconol as large particles and 8.3% wt amodimethicone as smaller particles. Particle size of the emulsion (E12) was determined using a Malvern Mastersizer®, for only the larger particles with Mode 2=6.698 μm. The Dv50 of the initial emulsion (B), as measured by a Microtrac Nanotrac 150, is of 0.0146 μm. These smaller particles cannot effectively be measured when the larger particles are present, because of the optical effect of the larger particles on the measurement apparatus. Nevertheless, evidence of the presence of such smaller particles is made by the flowable character of Emulsion 12 which would not be the case if only large particles were present at such high silicone content.

### Emulsion 13

Emulsification of (A) as a mixture of dimethicone 600 Pa·s and amodimethicone, with an emulsion (B) divinyldimethicone/dimethicone copolymer and C12-13 pareth-23 and C12-13 pareth-3 (B—60% silicone): 3.60 g of emulsion (B), 39.84 g of dimethicone 600 Pa·s, 9.96 g of amodimethicone and 1.20 g of water were weighed into a cup. The cup was closed and placed inside a DAC-150 SpeedMixer® and the cup was spun at maximum speed (3450 RPM) for 30 seconds. A first additional 5.40 g of emulsion (B) was added and the mixture was spun for 30 seconds at 3450 RPM. A further additional 5.00 g of emulsion (B) was added and the mixture was spun for 30 seconds at 3450 RPM. A last additional 5.80 g of emulsion (B) was added and the mixture was spun for 30 seconds at 3450 RPM.

The resulting emulsion (E13) consisted of an aqueous oil-in-water (o/w) emulsion of silicone polymer having a total silicone content of approximately 86.81% wt. On a dry basis, this emulsion (E13) contained approximately 64.8% wt dimethicone, 16.2% wt amodimethicone, and 19.0% wt divinyldimethicone/dimethicone copolymer. Particle size of the large emulsion particles (E13) was determined using a Malvern Mastersizer®; the large particles had Dv50=12.968 μm. Particle size of the small emulsion particles (E) was determined using a Microtrac Nanotrac 150; the small particles had Dv50=0.683 μm.

Emulsions 9 to 13 may be used in hair care applications such as colouring shampoos, permanent or semi-permanent colouring creams, styling aids, shampoos, rinse off conditioners, leave on conditioners and masks. Other applications of such emulsions in hair care will be apparent for the skilled in the art.

### COSMETIC COMPOSITION EXAMPLES

### Example 5

### O/W Emulsion

| | Ingredients of Example 5: O/W emulsion | % wt |
|---|---|---|
| | Phase A | |
| 1 | Glyceryl Stearate (and) PEG-100 Stearate | 4.0 |
| 2 | Mineral Oil | 8.0 |
| 3 | Simmondsia Chinensis (Jojoba) Seed Oil | 2.0 |
| 4 | Cyclopentasiloxane (and) Cyclohexasiloxane | 10.0 |
| 5 | Cetyl Alcohol | 1.0 |
| | Phase B | |
| 6 | Carbomer | 0.1 |
| 7 | Distilled Water | To 100.0% wt |
| 8 | Glycerin | 2.0 |
| 9 | Emulsion 8 | 6.4 |
| | Phase C | |
| 10 | Triethanolamine | 0.2 |
| 11 | Propylene Glycol (and) Diazolidinyl Urea (and) | q.s. |
| | Methylparaben (and) Propylparaben | |
| | Phase D | |
| 12 | Tocopheryl Acetate | 1.0 |
| 13 | Chitosan Succinamide | 1.0 |

Procedure to prepare the O/W emulsion of Example 5
1. Dilute ingredient 10 in 5 g of ingredient 7.
2. Melt ingredients 1 and 5 in ingredients 2 and 3.
3. Add Ingredient 4.
4. Disperse ingredient 6 in the rest of ingredient 7.
5. Add ingredients 8 and 9.
6. Add phase B to phase A with mixing.
7. Add mixture of ingredient 10 and 5 g of ingredient 7.
8. Add ingredient 11.
9. Add phase D and mix.
10. Homogenize using a high shear mixer.

### Example 6

### Foaming Cleanser

| | Ingredients of Example 6: foaming cleanser | % wt |
|---|---|---|
| | Phase A | |
| 1 | Simmondsia Chinensis (Jojoba) Seed Oil | 1.0 |
| 2 | Sodium Laureth Sulfate | 6.0 |
| 3 | Cocamidopropyl Hydroxysultaine | 3.0 |
| 4 | Decyl Glucoside | 5.0 |
| | Phase B | |
| 5 | Acrylates Copolymer | 8.0 |
| 6 | Emulsion 8 | 8.0 |
| 7 | Rubus Idaeus (Raspberry) Fruit Water | 20.0 |
| 8 | Distilled Water | To 100.0% wt |
| | Phase C | |
| 9 | Sodium Hydroxide (10% wt solution in water) | 1.7 |
| | Phase D | |
| 10 | Chitosan Succinamide | 1.0 |
| 11 | Propylene Glycol (and) Diazolidinyl Urea (and) | q.s. |
| | Methylparaben (and) Propylparaben | |

Procedure to prepare the foaming cleanser of Example 6
1. Mix Phase B ingredients together.
2. Add ingredient 2 with mixing.
3. Add ingredient 9 with mixing.
4. Add ingredients 1, 3 and 4 with mixing.
5. Add ingredients 10 and 11 and mix.

### Example 7

### Hydrogel

| | Ingredients of Example 7: hydrogel | % wt |
|---|---|---|
| | Phase A | |
| 1 | Emulsion 7 | 10.00 |
| 2 | Cyclopentasiloxane | 13.00 |
| 3 | Acrylates/C10-30 Alkyl Acrylate Crosspolymer | 0.60 |
| 4 | Distilled Water | To 100.00% wt |
| | Phase B | |
| 5 | Distilled Water | 5.00 |
| 6 | Triethanolamine | 0.13 |
| | Phase C | |
| 7 | Preservative | q.s. |

Procedure to prepare the hydrogel of Example 7
1. Prepare solution of ingredient 3 in ingredient 4.
2. Mix phase B ingredients together.
3. Combine phase A ingredients and mix.
4. Add phase B to phase A with mixing.
5. Add phase C with mixing.

### Example 8

### O/W Foundation

| | Ingredients of Example 8: O/W foundation | % wt |
|---|---|---|
| | Phase A | |
| 1 | Stearic Acid | 3.00 |
| 2 | Glyceryl Stearate (and) PEG-100 Stearate | 2.00 |
| 3 | Caprylic/Capric Triglyceride | 3.00 |
| 4 | Limnanthes Alba (meadow foam) Seed Oil | 2.40 |
| 5 | Mineral Oil | 1.00 |
| 6 | Ethylhexyl Methoxycinnamate | 3.00 |
| 7 | Dimethicone | 5.00 |
| 8 | Phenyl Trimethicone | 2.00 |
| | Phase B | |
| 9 | Distilled Water | To 100.00% wt |
| 10 | Emulsion 8 | 10.00 |
| 11 | Sodium Acrylates Copolymer (and) Glycine Soja | 0.25 |
| | (Soybean Oil) (and) PPG-1 Trideceth-6 | |
| 12 | Butylene Glycol | 8.00 |
| 13 | Titanium Dioxide | 9.50 |
| 14 | Iron Oxides Yellow | 1.50 |
| 15 | Iron Oxides Red | 0.35 |
| 16 | Iron Oxide Black | 0.10 |
| 17 | Talc | 1.00 |
| 18 | Triethanolamine | 0.90 |
| 19 | Propylene Glycol (and) Diazolidinyl Urea (and) | q.s. |
| | Methylparaben (and) Propylparaben | |

Procedure to prepare the O/W foundation of Example 8
1. Add ingredient 12 to ingredients 13, 14, 15, 16 and 17 to form pigment mix. Mix and then mill three times in 3-roll-mill
2. Combine phase A ingredients and mix.
3. Heat to 80° C.
4. Heat distilled water to 85° C.
5. Add ingredient 18 to distilled water.
6. Add phase A to distilled water and shear at high speed.
7. Reduce the shear and add pigment mix.
8. Mix for 5 minutes.
9. Add ingredient 11.
10. Add ingredient 10.

### Example 9

### Bi-Phase Leave-In Conditioner

| | Ingredients of Example 9: bi-phase | |
|---|---|---|
| | leave-in conditioner | % wt |
| | Phase A | |
| 1 | Panthenol | 0.30 |
| 2 | Distilled water | To 100.00% wt |
| 3 | Glycerin | 1.00 |
| 4 | Cetrimonium Chloride | 3.00 |
| 5 | Polyquaternium-11 | 0.30 |
| 6 | Cocamide MEA | 0.50 |
| 7 | Emulsion 9 | 10.00 |
| 8 | Amodimethicone (and) Trideceth-12 (and) | 5.00 |
| | Cetrimonium Chloride | |
| 9 | Methylchloroisothiazolinone (and) | 0.10 |
| | Methylisothiazolinone | |
| | Phase B | |
| 10 | Propylene Glycol | 2.00 |
| 11 | Perfume | 0.40 |
| | Phase C | |
| 12 | Aminopropyl Phenyl Trimethicone | 0.50 |
| 13 | Cyclopentasiloxane (and) Dimethiconol | 1.00 |

Procedure to prepare the bi-phase leave-in conditioner of Example 9
1. Mix ingredients of phase A together until homogeneous.
2. Mix ingredients of phase B together.
3. Add phase B to phase A with mixing.
4. Mix phase C and add to phase AB.
5. Mix until a homogeneous solution is obtained.

### Example 10

### W/O Emulsion

| | Ingredients of Example 10: W/O emulsion | % wt |
|---|---|---|
| | Phase A | |
| 1 | Cyclopentasiloxane (and) PEG/PPG-18/18 | 10.0 |
| | Dimethicone | |
| 2 | Cyclopentasiloxane | 15.5 |
| 3 | Cyclopentasiloxane (and) Trimethylsiloxysilicate | 3.0 |
| | Phase B | |
| 4 | Emulsion 7 | 2.3 |
| 5 | Sodium chloride | 2.0 |
| 6 | Distilled Water | To 100% wt |

Procedure to prepare the W/O emulsion of Example 10
1. Mix ingredients of phase A together.
2. Mix ingredient of phase B together.
3. Add phase B slowly (drop by drop) to phase A with continuous agitation.
4. Homogenize using a high shear mixer.

### Example 11

### Sunscreen and Self Tanning Cream

| | Ingredients of Example 11: sunscreen and self tanning cream | % wt |
|---|---|---|
| | Phase A | |
| 1 | Octocrylene | 2.00 |
| 2 | Ethylhexyl Salicylate | 2.00 |
| 3 | Phenyl Trimethicone | 6.00 |
| 4 | Cetyl Dimethicone | 2.00 |
| 5 | Caprylic/Capric Triglyceride | 3.00 |
| 6 | Sorbitan Stearate | 4.50 |
| 7 | Ceteth-20 | 2.50 |
| 8 | Ethylhexyl Methoxycinnamate | 4.00 |
| 9 | Polysorbate 80 | 1.50 |
| | Phase B | |
| 10 | Distilled water | 31.14 |
| 11 | Glycereth-26 | 4.00 |
| 12 | Hydroxyethylcellulose | 0.80 |
| | Phase C | |
| 13 | Distilled water | 20.96 |
| 14 | Dihydroxyacetone | 5.00 |
| | Phase D | |
| 15 | Cyclopentasiloxane | 6.00 |
| 16 | Emulsion 8 | 3.00 |
| 17 | Oleoyl Tyrosine (and) Butylene Glycol (and) Oleic Acid | 1.00 |
| 18 | Phenoxyethanol (and) Methylparaben (and) Butylparaben | 0.15 |
| | (and) Ethylparaben (and) Propylparaben (and) | |
| | Isobutylparaben | |
| 19 | BHT | 0.15 |
| | Phase E | |
| 20 | Perfume | 0.30 |

Procedure to prepare the sunscreen and self tanning cream of Example 11
1. Mix phase B ingredients together and heat to 80° C.
2. Combine phase A ingredients in order.
3. Heat phase A ingredients to 70-75° C.
4. Add phase B to phase A with mixing.
5. Cool to 45° C.
6. Mix phase C ingredients together.
7. Add phase C to A/B and homogenize.
8. Add phase D ingredients in order listed under stiffing and homogenize until uniform.
9. Add fragrance and mix well.

### Example 12

### Rinse Off Conditioner

| | Ingredients of Example 12: rinse off conditioner | % wt |
|---|---|---|
| | Phase A | |
| 1 | Cetearyl Alcohol | 4.5 |
| 2 | Behentrimonium Chloride | 1.5 |
| | Phase B | |
| 3 | Distilled water | 82.0 |
| 4 | EDTA | 0.1 |
| 5 | PEG-32 | 1.0 |
| 6 | Polyquaternium-10 | 0.3 |
| 7 | Guar Hydroxypropyltrimonium Chloride | 0.2 |
| | Phase C | |
| 8 | Propylene Glycol | 2.0 |
| 9 | Piroctone Olamine | 0.5 |
| 10 | Menthol | 0.3 |
| | Phase D | |
| 11 | Dimethicone (and) Bis-Hydroxy/Methoxy Amodimethicone | 2.0 |
| 12 | Emulsion 11 | 2.0 |
| 13 | Zinc Pyrithione | 2.0 |
| 14 | Water, matricaria, marigold and meadowsweet flowers | 0.3 |
| 15 | Propylene Glycol (and) Diazolidinyl Urea (and) | 0.5 |
| | Methylparaben (and) Propylparaben | |
| 16 | Perfume | q.s. |

Procedure to prepare the rinse off conditioner of Example 12
1. Mix phase A ingredients together and heat to 80° C.
2. Mix phase B ingredients together and heat to 80° C.
3. Add phase A to phase B with high shear mixing.
4. Gradually cool to 45° C. while stiffing.
5. Mix Phase C ingredients until completely dissolved. Add the mixture of Phase C into Phase AB.
6. Add Phase D ingredients one at a time in the mixture of ABC.
7. Mix until homogeneous.

### Example 13

### AP/Deo Roll On

| | Ingredients of Example 13: AP/deo roll on | % wt |
|---|---|---|
| | Phase A | |
| 1 | Distilled water | 41.0 |
| 2 | Steareth-20 | 0.6 |
| | Phase B | |
| 3 | Behentrimonium Methosulfate (and) Cetearyl Alcohol | 1.0 |
| 4 | Steareth-2 | 1.4 |
| 5 | PPG-3 Myristyl Ether | 3.0 |
| | Phase C | |
| 6 | Distilled water | 15.0 |
| 7 | Aluminum Zirconium Tetrachlorohydrex GLY | 30.0 |
| | Phase D | |
| 8 | Emulsion 7 | 3.0 |
| 9 | Distilled water | 5.0 |

Procedure to prepare the AP/deo roll on of Example 13
1. Mix ingredients of phase A together.
2. Mix ingredients of phase B together.
3. Separately heat phase A and Phase B to 70° C.
4. Add phase B to phase A with mixing.
5. Cool to 50° C.
6. Mix ingredients of phase C together.
7. Add phase C to the previous mix. Cool to room temperature.
8. Mix ingredients of phase D together.
9. Add phase D with slow mixing.

1. A cosmetic composition comprising:
a bi-modal water continuous emulsion (E) comprising at least 70 weight percent of: a first dispersed phase containing a hydrophobic oil, wherein the hydrophobic oil is provided as a non-emulsified hydrophobic oil, and a second dispersed phase containing a silicone, wherein the silicone is provided from a water continuous silicone emulsion containing at least one surfactant;
and at least one cosmetic ingredient (C),
optionally in a cosmetically acceptable medium.

2. The cosmetic composition of claim 1, where the hydrophobic oil of the first dispersed phase is selected from an organic oil, a silicone, or combinations thereof.

3. The cosmetic composition of claim 1, where the dispersed phase containing a silicone is selected from a “macro” emulsion or a “micro” emulsion.

4. The cosmetic composition of claim 1, where the cosmetic ingredient (C) is selected from emollients, waxes, moisturizers, surface active materials, thickeners, water phase stabilizing agents, pH controlling agents, preservatives and cosmetic biocides, sebum absorbants, sebum control agents, vegetable extracts, botanical extracts, vitamins, proteins and their derivatives, amino-acids and their derivatives, pigments, colorants, fillers, silicone conditioning agents, cationic conditioning agents, UV absorbers, sunscreen agents, antidandruff agents, antiperspirant agents, deodorant agents, skin protectants, hair dyes, nail care ingredients, fragrances, perfume, antioxidants, oxidizing agents, reducing agents, film formers, propellant gases, fatty alcohols, color care additives, pearlising agents, chelating agents, film formers, styling agents, ceramides, suspending agents, and mixtures thereof.

5. The cosmetic composition of claim 1, where the cosmetic ingredient (C) is selected from a cationic conditioning agent, a hydrophobic conditioning agent, and mixtures thereof.

6. The cosmetic composition of claim 1, which is in the form of a cream, a gel, a powder (free flowing powder or pressed), a paste, a solid, a freely pourable liquid, or an aerosol.

7. The cosmetic composition of claim 1, which is in the form of a shampoo, a cream, a rinse-off conditioner, a leave-in conditioner, or a gel.

8. A process for preparing the cosmetic composition of claim 1, the process comprising the step of:
mixing the bi-modal water continuous emulsion (E); comprising at least 70
and the cosmetic ingredient (C),
optionally in the presence of a cosmetically acceptable medium.

9. (canceled)

10. A process of washing keratinous fibres comprising the steps of:
mixing a bi-modal water continuous emulsion (E) comprising at least 70 weight percent of: a first dispersed phase containing a hydrophobic oil, wherein the hydrophobic oil is provided as a non-emulsified hydrophobic oil, and a second dispersed phase containing a silicone, wherein the silicone is provided from a water continuous silicone emulsion containing at least one surfactant;
with at least one cosmetic ingredient (C), optionally in the presence of a cosmetically acceptable medium, to form a mixture; and
applying the mixture to the keratinous fibres; and
optionally letting the mixture stand on the keratinous fibres; and/or
optionally rinsing the keratinous fibres.

11. A process of conditioning keratinous fibres comprising the steps of:
mixing a bi-modal water continuous emulsion (E) comprising at least 70 weight percent of: a first dispersed phase containing a hydrophobic oil, wherein the hydrophobic oil is provided as a non-emulsified hydrophobic oil, and a second dispersed phase containing a silicone, wherein the silicone is provided from a water continuous silicone emulsion containing at least one surfactant;
with at least one cosmetic ingredient (C) selected from a cationic conditioning polymer, a hydrophobic conditioning polymer, and mixtures thereof, optionally in the presence of a cosmetically acceptable medium, to form a mixture; and
applying the mixture to the keratinous fibres; and
optionally letting the mixture stand on the keratinous fibres; and/or
optionally rinsing the keratinous fibres.

12. (canceled)

13. The cosmetic composition of claim 1, where the cosmetically acceptable medium is present.

14. The cosmetic composition of claim 2, where the dispersed phase containing a silicone is selected from a “macro” emulsion or a “micro” emulsion.

15. The cosmetic composition of claim 2, where the hydrophobic oil of the first dispersed phase comprises the organic oil, with the organic oil selected from the group of hydrocarbons, esters, oils derived from natural fats or natural oils, organic polymers, or mixtures thereof.

16. The cosmetic composition of claim 2, where the hydrophobic oil of the first dispersed phase comprises the silicone.

17. A process of washing and/or conditioning keratinous fibres, the process comprising the step of applying a cosmetic composition to the keratinous fibres, wherein the cosmetic composition is as set forth in claim 1.
